(19) United States
(12) Patent Application Publication
(10) Pub. No.: US 2003/0162824 A1
(43) Pub. Date: Aug. 28, 2003

(54) Methods of treating or preventing a cardiovascular condition using a cyclooxygenase-1 inhibitor

(76) Inventor: Elaine S. Krul, Warson Woods, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/292,255

(22) Filed: Nov. 12, 2002

Related U.S. Application Data
(60) Provisional application No. 60/338,291, filed on Nov. 13, 2001.
(60) Provisional application No. 60/331,346, filed on Nov. 12, 2001.

(51) Int. Cl. A61K 31/415

(52) U.S. Cl. 514/406

(57) ABSTRACT

Methods for treating or preventing one or more cardiovascular conditions in a subject comprising treating the subject with a therapeutically effective amount of a selective cyclooxygenase-1 inhibitor or a pharmaceutically-acceptable salt, tautomer or prodrug thereof alone or in combination with either a drug used in the treatment or prevention of a cardiovascular condition or a non-drug therapy used in the treatment of a cardiovascular condition.

### CROSS-REFERENCE TO RELATED APPLICATIONS

This application claims the benefit of U.S. Provisional Application No. 60/338,291, filed Nov. 13, 2001, and U.S. Provisional Application No. 60/331,346, filed Nov. 12, 2001, both of which are hereby incorporated by reference in their entirety.

### FIELD OF THE INVENTION

This invention is in the field of treating or preventing cardiovascular conditions. More specifically, this invention relates to the use of selective cyclooxygenase-1 inhibitors or derivatives thereof in treating or preventing cardiovascular conditions including atherosclerosis.

### BACKGROUND OF THE INVENTION

Recent research efforts have focused on the role of inflammation in the development and progression of cardiovascular conditions. For example, Ridker et al. (New Eng. J. Med., 336, 973-9 (1997)), discusses a possible role of inflammation in cardiovascular disease. Similarly, J. Boyle (J. Path., 181,93-9 (1997)), describes a possible relationship between plaque rupture and atherosclerotic inflammation.

Prostaglandins play a major role in the inflammation process and the inhibition of prostaglandin production (especially production of PGG₂, PGH₂, and PGE₂) has been a target of anti-inflammatory drug discovery. Until recently, most available non-steroidal anti-inflammatory drugs (“NSAIDs”) that were active in reducing the prostaglandin-induced pain and swelling associated with the inflammation process also were active in affecting other prostaglandin-regulated processes not associated with the inflammation process. Use of high doses of such NSAIDs often produced severe side effects, including life-threatening ulcers, and limited the therapeutic use of these NSAIDs. One alternative to such NSAIDs was the use of corticosteroids. Use of corticosteroids, however, also often produced severe adverse effects, especially when long-term therapy was involved.

In recent years, a class of NSAIDs has been discovered that prevents the production of prostaglandins by inhibiting the cyclooxygenase (“COX”) enzyme in the human arachidonic acid/prostaglandin pathway. Two isoforms of the cyclooxygenase enzyme that are associated with inflammation have been identified, cyclooxygenase-1 (“COX-1”, also known as “prostaglandin G/H synthase I”) and cyclooxygenase-2 (“COX-2”, also known as “prostaglandin G/H synthase 11”). The cyclooxygenase-2 enzyme provides a target for inhibition that more effectively reduces inflammation and produces fewer and less drastic side effects in comparison to traditional NSAIDs. Recent anti-inflammation drugs such as CELEBREX® and VIOXX® are compounds that selectively inhibit the cyclooxygenase-2 enzyme relative to the cyclooxygenase-1 enzyme. Inhibition of the cyclooxygenase-1 enzyme to treat inflammation, however, was thought undesirable because cyclooxygenase-1 is also involved in other regulatory processes not associated with inflammation such as processes associated with maintaining the gastrointestinal lining. Such inhibition was generally expected to result in adverse side-effects including severe gastrointestinal disorders.

Published application WO 01/28548 recently disclosed the use of a cyclooxygenase-2 inhibiting compound to prevent heart disease. Similarly, published application WO 98/47509 discloses the use of a cyclooxygenase-2 inhibiting compound to prevent inflammation-related cardiovascular disorders such as atherosclerosis.

Compounds that inhibit cyclooxygenase-1 have been described in the literature. See, for example, published application WO 98/57910. Likewise, compounds that selectively inhibit cyclooxygenase-1 have been described in the literature. See, for example, the group of 3-(trifluoromethyl)-1,5-diarylpyrazoles described in Journal of Medicinal Chemistry, vol. 40, pages 1347-65 (Penning, et al.). The thiazole compound FR1 22047 (1-[(4,5-bis(4-methoxyphenyl)-2-thiazoyl)carbonyl]-4-methyl piperazine hydrochloride), originally disclosed in U.S. Pat. No. 5,217,971, also has been reported to be a selective cyclooxygenase-1 inhibitor by Ochi, et al. European Journal of Pharmacology, vol. 391, pages 49-54 (2000). The use of such cyclooxygenase-1 inhibiting compounds to treat cardiovascular-related conditions, however, has not been previously described.

Efforts continue to develop additional drug therapies that can be used to treat or prevent cardiovascular conditions. Accordingly, the present application discloses one such newly discovered drug therapy. Specifically, it has been discovered selective cyclooxygenase-1 inhibiting compounds can be administered to treat or prevent inflammation related cardiovascular conditions.

The use of such selective cyclooxygenase-1 inhibiting compounds in this manner was not previously disclosed in the literature.

### SUMMARY OF THE INVENTION

The invention provides, in a first aspect, methods for treating or preventing an inflammation-related cardiovascular condition in a subject comprising administering to the subject a therapeutically effective amount of a selective cyclooxygenase-1 inhibiting compound. In a second aspect, the invention provides methods for treating or preventing an inflammation-related cardiovascular condition in a subject comprising administering to the subject a therapeutically-effective amount of a compound of Formula I:

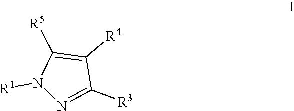

wherein:

R¹ is substituted or unsubstituted aryl;

one of R³ and R⁴ is hydrogen, and the other of R³ and R⁴ is an electron withdrawing group or a substituted or unsubstituted alkyl group; and

R⁵ is substituted or unsubstituted aryl;

or R³ is an electron withdrawing group or a substituted or unsubstituted alkyl group, and R⁴ and R⁵ combine to form a fused ring substituent; or a pharmaceutically acceptable salt, tautomer, or prodrug thereof.

In a third aspect, the invention provides methods for treating or preventing an inflammation-related cardiovascular condition in a subject comprising administering to the subject a selective cyclooxygenase-1 inhibiting compound in combination with either other active drugs used in the treatment or prevention of cardiovascular-related conditions, or non-drug therapies used in the treatment of cardiovascular-related conditions, such that the combination therapy is therapeutically effective in the treatment or prevention of the inflammation-related cardiovascular condition.

Other aspects of the invention are discussed throughout the specification of this application.

### DETAILED DESCRIPTION OF THE PREFERRED EMBODIMENTS

The present invention provides methods for treating or preventing one or more cardiovascular conditions in a subject. The methods comprise treating the subject with a therapeutically effective amount of a selective cyclooxygenase-1 inhibitor or a pharmaceutically-acceptable salt, tautomer or prodrug thereof.

The present methods are useful for, but not limited to, treating or preventing in a subject an inflammation-related cardiovascular condition selected from the group consisting of coronary artery disease, aneurysm, arteriosclerosis, atherosclerosis (including cardiac transplant atherosclerosis), myocardial infarction, heart failure, embolism, stroke, aortic stenosis, renal dysfunction, renal failure, microalbuminuria, thrombosis (including venous thrombosis), angina (including unstable angina), myocarditis, pericarditis, coronary plaque inflammation, bacterial-induced inflammation (including Ch/amydia-induced inflammation), viral induced inflammation, auto-immune-induced inflammation, and inflammation and/or hyperplasia associated with surgical procedures such as vascular grafting including coronary artery bypass surgery, revascularization procedures including angioplasty, stent placement, endarterectomy, or other invasive procedures involving arteries, veins and capillaries. Cardiovascular conditions likewise may include, but are not limited to, those conditions that have an inflammation component and those that may be mediated by cyclooxygenase-1.

These methods are useful for human subjects as well as mammalian companion animals, exotic animals and farm animals and the like. The subject preferably is a mammal such as a human, dog, cat or horse, and most preferably is a human.

The present invention also provides a combination therapy for treating or preventing an inflammation-related cardiovascular condition in a subject, the methods comprising treating the subject with a selective cyclooxygenase-1 inhibitor or a pharmaceutically-acceptable salt, tautomer or prodrug thereof and either another active drug used in the treatment or prevention of cardiovascular-related conditions or a non-drug therapy used in the treatment of a cardiovascular condition, such that the combination therapy is therapeutically effective in the treatment or prevention of the inflammation-related cardiovascular condition.

The present invention further provides kits useful in the treatment or prevention of inflammation-related cardiovascular conditions.

Definitions

The term “administration” in defining the use of both a selective cyclooxygenase-1 inhibitor and another drug used in the treatment or prevention of cardiovascular-related conditions is intended to embrace administration of each agent in a manner and in a regimen that will provide beneficial effects of the drug combination therapy, and is intended as well to embrace co-administration of 2 or more of the COX-1 agents in a substantially simultaneous manner and/or 2 or more of the drugs used in the treatment or prevention of cardiovascular-related conditions in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from the constituent COX-1 agent and drug used in the treatment or prevention of cardiovascular-related conditions when used in combination.

The term “angioplasty” includes the alteration of the structure of a vessel, either by dilating the vessel using a balloon inside the lumen or by other surgical procedure. The term “angioplasty” includes, but is not limited to, percutaneous transluminal coronary angioplasty.

The term “combination therapy” (or “co-therapy”), when used with respect to drug combinations, is intended to embrace the administration of each agent in a sequential manner in a regimen that will provide beneficial effects of the drug combination, and is intended as well to embrace co-administration of these agents in a substantially simultaneous manner, such as in a single capsule or injection having a fixed ratio of these active agents or in multiple, separate capsules or injections for each agent. The terms “cyclooxygenase-1 selective inhibitor,” “COX-1 selective inhibitor,” and “selective cyclooxygenase-1 inhibiting compound” interchangeably refer to a therapeutic compound that selectively inhibits the cyclooxygenase-1 isoform of the enzyme cyclooxygenase. For the purposes of this application, cyclooxygenase-1 selectivity can be measured as a ratio of the in vitro or in vivo IC₅₀ value for inhibition of cyclooxygenase-2 divided by the corresponding IC₅₀ value for inhibition of cyclooxygenase-1. In practice, however, cyclooxygenase-1 selectivity varies depending on the conditions under which the test is performed and on the inhibitors being tested. One illustrative test for these measuring IC₅₀ values is set forth in the working examples of this application. A cyclooxygenase-1 selective inhibitor is any inhibitor for which the ratio of cyclooxygenase-2 IC₅₀ to cyclooxygenase-1 IC₅₀ is greater than 1, preferably greater than 5, more preferably greater than 10, still more preferably greater than 50, and more preferably still greater than 100.

Even more preferred are compounds that satisfy one or both of the following conditions: (a) the compound has a cyclooxygenase-1 IC₅₀ of less than about 1 μM, preferably less than about 0.5 μM, and more preferably less than about 0.2 μM, and (b) the compound has a cyclooxygenase-2 IC₅₀ of greater than about 1 μM, preferably greater than about 5 μM, and more preferably greater than about 10 μM.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “prevention” includes either preventing the onset of a clinically evident cardiovascular condition altogether or preventing the onset of a preclinically evident stage of a cardiovascular condition in a subject. This term encompasses the prophylactic treatment of a subject at risk of developing a cardiovascular condition, including but not limited to atherosclerosis.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

The term “restenosis” includes the reoccurrence of a stenosis condition in the vasculature of a subject, particularly the reoccurrence of a stenosis condition treated by angioplasty.

The term “subject” for purposes of treatment includes any human or animal subject who is suffering from or susceptible to any one of the known cardiovascular conditions, and preferably is a human subject. The subject may be at risk due to diet, exposure to bacterial or viral infection, having common markers present, being genetically predisposed to the cardiovascular conditions, and the like.

The term “surgery” is used in accordance with its ordinary meaning and should be interpreted broadly. It includes, but is not limited to, surgery such as angioplasty, arterial resection, tissue reconstruction, tissue graft including but not limited to venous, arterial and prosthetic materials, treatment of fractures and other damage to bones, repair of an arterioyenuous fistula, and digital replantation.

The term “therapeutically effective” is intended to qualify the amount of each agent which will achieve the goal of prevention of disease or improvement in condition severity or the frequency of incidence over treatment of each agent by itself, while avoiding adverse side effects typically associated with alternative therapies.

The term “trauma” includes a physical injury or wound caused by external force or violence including, but not limited to, injury resulting from a motor vehicle accident, a suicide attempt, a fall, burns, amputation or the like.

The term “treatment” includes any process, action, application, therapy, procedure or the like, wherein a mammal, particularly a human, is subjected to medical aid with the object of improving the mammal's condition, directly or indirectly. Treatment also can include slowing or stopping the progression of a clinically evident cardiovascular condition altogether or slowing or stopping the progression of the onset of a preclinically evident stage of a cardiovascular condition in a subject.

The term “vascular injury” as used in this application includes damage to the vasculature of a subject resulting from and secondary to, for example, trauma, surgery or the like as distinguished from damage to the vasculature resulting solely from arteriosclerotic or atherosclerotic vascular disease.

Vascular injury also can result, for example, from an infarct. When there is a disruption of blood flow from, for example, a thrombus, ischemia from the disruption of blood flow can result in necrosis. A series of ventricular remodeling and cardiovascular events can ensue that involves mechanisms underlying restenosis, namely smooth muscle cell proliferation, cellular matrix production, and the like.

Where used, either alone or within other terms such as “haloalkyl”, “alkylsulfonyl”, “alkoxyalkyl” and “hydroxyalkyl”, the term “alkyl” embraces linear, cyclic or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “halo” means halogens such as fluorine, chlorine, bromine or iodine.

The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “carbonyl”, whether used alone or with other terms, such as “alkoxycarbonyl”, denotes —(C═O)—.

The terms “carboxy” or “carboxyl”, whether used alone or with other terms, such as “carboxyalkyl”, denotes —CO2H.

The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The term “electron withdrawing group” embraces substituent groups that exert an electron withdrawing effect, including perfluoroalkyl (e.g. —CF₃), —CF₂H, nitriles (—CN), substituted or unsubstituted amides, —COOR (wherein R═H, an alkyl, or an ester), —CO-alkyl, —SO₂-alkyl, hydroxy, or a halogen.

Illustrative Embodiments

In one embodiment, the present invention is directed to a method of treating or preventing in a subject an inflammation-related cardiovascular condition comprising administering a therapeutically effective amount of a selective COX-1 inhibitor to a mammalian subject susceptible to or suffering from an inflammation-related cardiovascular condition.

In another embodiment, the present invention provides a method of treating or preventing an inflammation-related cardiovascular condition in a subject susceptible to or suffering from such a condition, the method comprising administering to the subject a selective COX-1 inhibitor in combination with either a drug used for the treatment or prevention of a cardiovascular condition or a non-drug therapy used in the treatment of a cardiovascular condition, such that the combination therapy is therapeutically effective in the treatment or prevention of the inflammation-related cardiovascular condition.

Also provided is a kit for treating or preventing an inflammation-related cardiovascular condition, wherein the kit contains a first dosage form comprising a cyclooxygenase-1 selective inhibitor and a second dosage form comprising another active drug used in the treatment or prevention of cardiovascular conditions.

In a further embodiment, the drug used for the treatment or prevention of a cardiovascular condition is selected from the group consisting of renin inhibitors, angiotension I receptor antagonists, angiotension II receptor antagonists, angiotension converting enzyme inhibitors, alpha-adrenergic receptor blockers, beta-adrenergic receptor blockers, calcium channel blockers, neutral endopeptidase inhibitors, aldosterone receptor antagonists, vasodilators, diuretics, lipid-lowering drugs, anti-oxidants, and IIbIIIa antagonists.

The inflammation-related cardiovascular condition may be selected from the group consisting of coronary artery disease, aneurysm, arteriosclerosis, atherosclerosis including cardiac transplant atherosclerosis, myocardial infarction, heart failure, embolism, stroke, aortic stenosis, renal dysfunction, renal failure, microalbuminuria, thrombosis (including venous thrombosis), angina (including unstable angina), myocarditis, pericarditis, coronary plaque inflammation, bacterial-induced inflammation (including Chlamydia-induced inflammation), viral induced inflammation, auto-immune-induced inflammation, and inflammation and/or hyperplasia associated with surgical procedures such as vascular grafting including coronary artery bypass surgery, revascularization procedures including angioplasty, stent placement, endarterectomy, or other invasive procedures involving arteries, veins and capillaries. Cardiovascular conditions likewise may include, but are not limited to, those conditions that have an inflammation component and those that may be mediated by cyclooxygenase-1.

In another embodiment, the present invention is directed to a method for treating or preventing a cardiovascular condition, caused or aggravated by COX-1 activity in a subject. The method comprises administering a selective COX-1 inhibitor to a mammalian subject susceptible to or suffering from a cardiovascular condition, wherein the selective COX-1 inhibitor is administered in an amount that is therapeutically effective in suppressing the cardiovascular condition. In a further embodiment, the present invention is directed to a method for treating or preventing a cardiovascular condition, caused or aggravated by COX-1 activity in a subject, the method comprising administering a selective COX-1 inhibitor to a mammalian subject susceptible to or suffering from a cardiovascular condition, in combination with a drug used in the treatment or prevention of a cardiovascular condition, wherein the selective COX-1 inhibitor and drug used in the treatment or prevention of a cardiovascular condition are administered in amounts that are therapeutically effective in suppressing the cardiovascular condition.

In another embodiment the present invention is directed to a method for treating or preventing inflammation and/or hyperplasia resulting from vascular injury in a subject. The method comprise administering a selective COX-1 inhibitor to a mammalian subject susceptible to or suffering from inflammation and/or hyperplasia resulting from vascular injury, wherein the selective COX-1 inhibitor is administered in an amount that is therapeutically effective in suppressing the inflammation and/or hyperplasia. The vascular injury generally is caused by trauma or surgery and is an injury to a vessel such as an artery, preferably a coronary artery or a pulmonary artery. Preferably, the injury is to a coronary artery such as, but not limited to, injury substantially caused by angioplasty and substantially resulting in the occurrence of restenosis.

In another embodiment, the present invention is directed to a method for treating or preventing restenosis of a vessel, particularly an artery, resulting from vascular injury in a subject, preferably a human subject. The method comprises administering a selective COX-1 inhibitor in a mammalian subject susceptible to or suffering from said restenosis, wherein the selective COX-1 inhibitor is administered in an amount that is therapeutically effective in suppressing restenosis. The method of this embodiment preferably is used to treat restenosis resulting from trauma or surgery, particularly restenosis in a human subject who has undergone angioplasty of an artery, and more particularly restenosis substantially resulting from angioplasty of the coronary artery.

Cyclooxygenase-1 Inhibitors

The COX-1 inhibitors used in the present invention are selective COX-1 inhibitors. In one embodiment, the cyclooxygenase-1 selective inhibitor is a compound of Formula I:

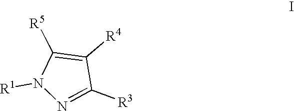

wherein:

R¹ is substituted or unsubstituted aryl;

one of R³ and R⁴ is hydrogen or a halogen, and the other of R³ and R⁴ is an electron withdrawing group or a substituted or unsubstituted alkyl group; and

R⁵ is substituted or unsubstituted aryl;

or R³ is an electron withdrawing group or a substituted or unsubstituted alkyl group, and R⁴ and R⁵ combine to form a fused ring substituent;

or a pharmaceutically acceptable salt, tautomer or prodrug thereof.

In another embodiment, the selective cyclooxygenase-1 inhibitor is a compound of Formula I wherein:

R¹ is substituted or unsubstituted phenyl;

one of R³ and R⁴ is hydrogen, and the other of R³ and R⁴ is selected from the group consisting of halogen, cyano, hydroxy, carboxy, haloalkyl, hydroxyalkyl, and alkoxycarbonyl; and

R⁵ is substituted or unsubstituted phenyl;

or a pharmaceutically acceptable salt, tautomer or prodrug thereof.

In another embodiment, the cyclooxygenase-1 selective inhibitor is a compound of Formula I wherein:

R¹ is phenyl, wherein said phenyl is unsubstituted or is substituted with one or more groups selected from the group consisting of halogen, alkyl, haloalkyl, alkoxy and alkylthio;

one of R³ and R⁴ is hydrogen, and the other of R³ and R⁴ is selected from the group consisting of halogen, cyano, hydroxy, carboxy, haloalkyl, hydroxyalkyl, and alkoxycarbonyl; and

R⁵ is phenyl, wherein said phenyl is unsubstituted or is substituted with one or more groups selected from the group consisting of halogen, alkyl, haloalkyl, alkoxy and alkylthio;

or a pharmaceutically acceptable salt, tautomer or prodrug thereof. In another embodiment, the cyclooxygenase-1 selective inhibitor is a compound of Formula I wherein:

R¹ is phenyl, wherein said phenyl is unsubstituted or is substituted with one or more groups selected from the group consisting of halogen, (C₁-C₆)alkyl, halo-(C₁-C₆)alkyl, (C₁-C₆)alkoxy, and (C₁-C₆)alkylthio;

one of R³ and R⁴ is hydrogen, and the other of R³ and R⁴ is selected from the group consisting of halogen, cyano, hydroxy, halo-(C₁-C₆)alkyl, hydroxy-(C₁-C₆)alkyl, and (C₁-C₆)alkoxycarbonyl; and

R⁵ is phenyl, wherein said phenyl is unsubstituted or is substituted with one or more groups selected from the group consisting of halogen, (C₁-C₆)alkyl, halo-(C₁-C₆)alkyl, (C₁-C₆)alkoxy, and (C₁-C₆)alkylthio;

or a pharmaceutically acceptable salt, tautomer or prodrug thereof.

In another embodiment, the cyclooxygenase-1 selective inhibitor is a compound of Formula I wherein:

R¹ is phenyl, wherein said phenyl is unsubstituted or is substituted with one or more groups selected from the group consisting of halogen, (C₁-C₂)alkyl, halo-(C₁-C₂)alkyl, (C₁-C₂)alkoxy, and (C₁-C₂)alkylthio;

one of R³ and R⁴ is hydrogen, and the other of R³ and R⁴ is selected from the group consisting of halogen, cyano, hydroxy, halo-(C₁-C₂)alkyl, hydroxy-(C₁-C₂)alkyl, and (C₁-C₂)alkoxycarbonyl; and

R⁵ is phenyl, wherein said phenyl is unsubstituted or is substituted with one or more groups selected from the group consisting of halogen, (C₁-C₂)alkyl, halo-(C₁-C₂)alkyl, (C₁-C₂)alkoxy, and (C₁-C₂)alkylthio;

or a pharmaceutically acceptable salt, tautomer or prodrug thereof.

In another embodiment, the cyclooxygenase-1 selective inhibitor is a compound of Formula I as described above wherein one or both of the R¹ and R⁵ phenyl substituents are independently mono-substituted at the para position, and the R³ substituent is hydrogen.

In another embodiment, the cyclooxygenase-1 selective inhibitor is a compound of Formula I as described above wherein one or both of the R¹ and R⁵ phenyl substituents are independently mono-substituted at the para position, and the R⁴ substituent is hydrogen.

In another embodiment, the cyclooxygenase-1 selective inhibitor is a compound of Formula I selected from the group consisting of the compounds listed in Table 1, and the pharmaceutically acceptable salts, tautomers and prodrugs thereof.

TABLE 1

| | | hCox-1 | hCox-2 |
|---|---|---|---|
| Compound | | Avg. | Avg. |
| Number | Compound Structure | IC50 (μM) | IC50 (μM) |
| 1 | 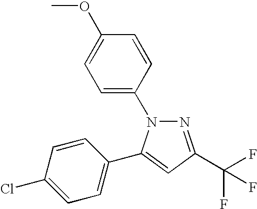 | <0.137 0.00100 | 49.8 >100 |
| | 5-(4-chlorophenyl)-1-(4-methoxyphenyl)-3-(trifluoromethyl)pyrazole | | |
| 2 | 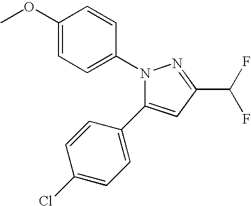 | <0.137 0.0210 | >100 >100 |
| | 5-(4-chlorophenyl)-3-(difluoromethyl)-1-(4-methoxyphenyl)pyrazole | | |
| 3 | 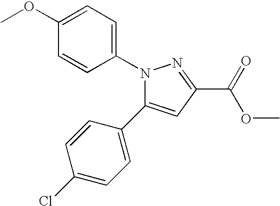 | 0.284 | >100 |
| | 5-(4-chlorophenyl)-3-(methoxycarbonyl)-1-(4-methoxyphenyl)pyrazole | | |
| 4 | 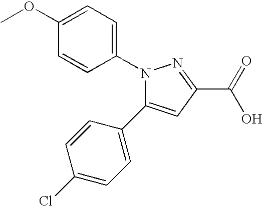 | 55.7 | >100 |
| | 3-carboxy-5-(4-chlorophenyl)-1-(4-methoxyphenyl)pyrazole | | |
| 5 | 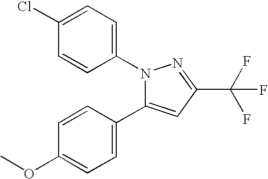 | 0.0180 | 0.746 |
| | 1-(4-chlorophenyl)-5-(4-methoxyphenyl)-3-(trifluoromethyl)pyrazole | | |
| 6 | 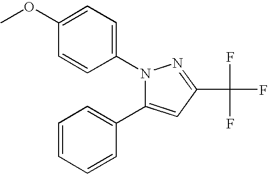 | 0.0180 | >1000 |
| | 1-(4-methoxyphenyl)-5-phenyl-3-(trifluoromethyl)pyrazole | | |
| 7 | 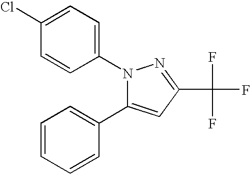 | 0.0850 | >1000 |
| | 1-(4-chlorophenyl)-5-phenyl-3-(trifluoromethyl)pyrazole | | |
| 8 | 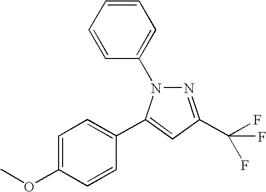 | 0.0960 | >1000 |
| | 5-(4-methoxyphenyl)-1-phenyl-3-(trifluoromethyl)pyrazole | | |
| 9 | 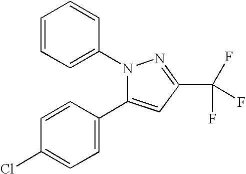 | 0.123 | >1000 |
| | 5-(4-chlorophenyl)-1-phenyl-3-(trifluoromethyl)pyrazole | | |
| 10 | 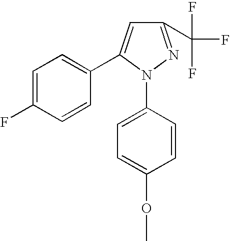 | | |
| | 5-(4-fluorophenyl)-1-(4-methoxyphenyl)-3-(trifluoromethyl)pyrazole | | |
| 11 | 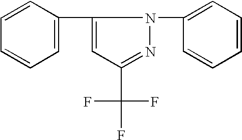 | 0.972 | >100 |
| | 1,5-diphenyl-3-(trifluoromethyl)pyrazole | | |
| 12 | 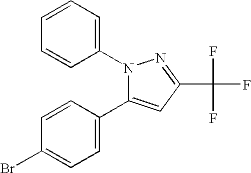 | 0.435 | >500 |
| | 5-(4-bromophenyl)-1-phenyl-3-(trifluoromethyl)pyrazole | | |
| 13 | 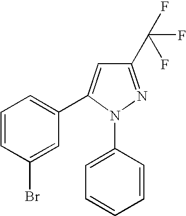 | 3.3 | >100 |
| | 5-(3-bromophenyl)-1-phenyl-3-trifluoromethyl)pyrazole | | |
| 14 | 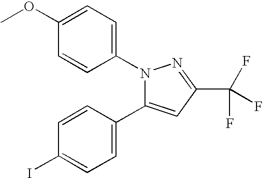 | <0.00100 | 1.54 |
| | 5-(4-iodophenyl)-1-(4-methoxyphenyl)-3-(trifluoromethyl)pyrazole | | |
| 15 | 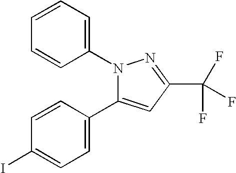 | 0.650 | >100 |
| | 5-(4-iodophenyl)-1-phenyl-3-(trifluoromethyl)pyrazole | | |
| 16 | 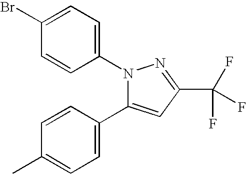 | <0.137 | 0.424 |
| | 1-(4-bromophenyl)-5-(4-methylphenyl)-3-(trifluoromethyl)pyrazole | | |
| 17 | 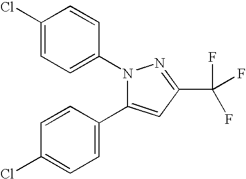 | 0.055 | 9.9 |
| | 1,5-bis(4-chlorophenyl)-3-(trifluoromethyl)pyrazole | | |
| 18 | 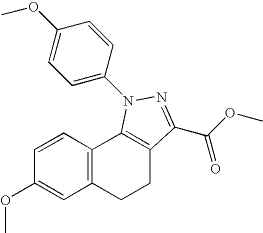 | 0.442 | >100 |
| 19 | 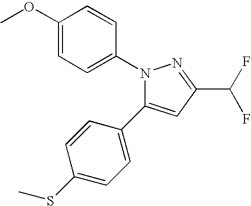 | 0.126 | >100 |
| 20 | 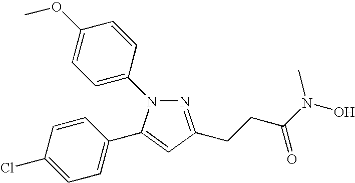 | 0.027 | >1000 |
| 21 | 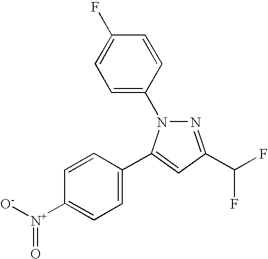 | 0.647 | 22.8 |
| 22 | 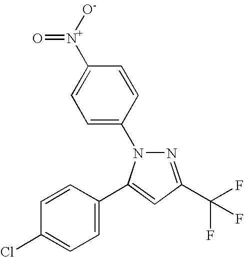 | 0.810 | >100 |
| 23 | 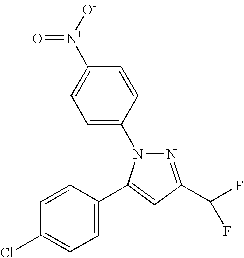 | 0.676 | >100 |
| 24 | 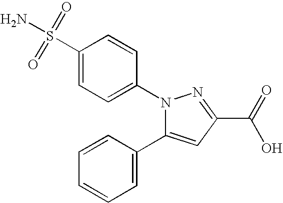 | 0.137 | 100 |
| 25 | 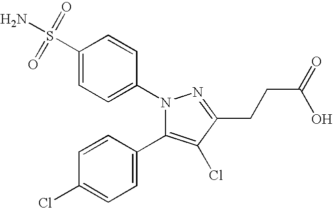 | 0.169 | 4.69 |
| 26 | 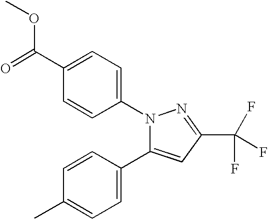 | 0.310 | >100 |
| 27 | 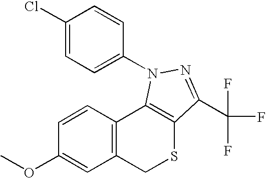 | 0.141 | >100 |
| 28 | 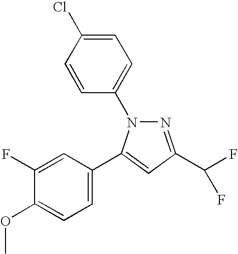 | 0.643 | 23 100 |
| 29 | 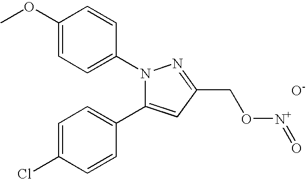 | 0.242 | 100 |
| 30 | 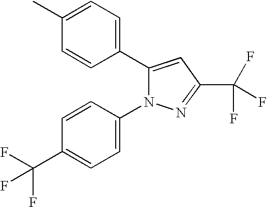 | 0.108 | >100 |
| 31 | 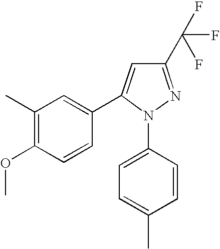 | 0.287 | 67.1 |
| 32 | 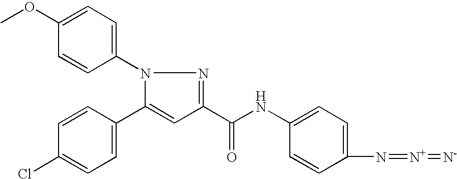 | 0.012 | 1000 |

In another embodiment, the cyclooxygenase-1 selective inhibitor is 1,5-bis(4-methoxyphenyl)-3-(trifluoromethyl)pyrazole or the pharmaceutically-acceptable salts, tautomers and prodrugs thereof.

In another embodiment, the cyclooxygenase-1 selective inhibitor is a 3-(trifluoromethyl)-1,5-diarylpyrazole, or a pharmaceutically-acceptable salt, tautomer or prodrug thereof. Preferably, the compound does not comprise a sulfonamide moiety.

In another embodiment, the cyclooxygenase-1 selective inhibitor is a 3-(difluoromethyl)-1,5-diarylpyrazole, or a pharmaceutically acceptable salt, tautomer, or prodrug thereof. Preferably, the compound does not comprise a sulfonamide moiety.

The compounds utilized in the methods of the present invention may be present in the form of free bases or pharmaceutically acceptable acid addition salts thereof. The term “pharmaceutically-acceptable salts” embraces, for example, salts commonly used to form alkali metal salts and to form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, gamma-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding cyclooxygenase-1 inhibitor compounds.

Biological Evaluation

The efficacy of a selective cyclooxygenase-1 inhibiting compound can be evaluated using, for example, an appropriate in vivo model. Several illustrative animal models that are available and that can be used to evaluate the treatment or prevention of a cardiovascular condition (including atherosclerosis) include, but are not limited to, the following:

(1) The model disclosed in Stehbens, Prog. Card. Dis., XXIX, 1007-28 (1986) and Zhang et al., Science, 258, 468-71 (1992); and

(2) The APOe mouse model for atherosclerosis described by Roselear et al. (Arterioscle. Thromb. Vasc. Biol., 16,1013-18 (1996)).

Pharmaceutical Compositions

The present invention also is directed to pharmaceutical compositions comprising a cyclooxygenase-1 inhibitor that can be used to treat or prevent a cardiovascular condition in a subject. The composition comprises a therapeutically-effective amount of a selective cyclooxygenase-1 inhibiting compound in association with at least one pharmaceutically-acceptable carrier, adjuvant or diluent (collectively referred to herein as “carrier” materials) and, if desired, other active ingredients.

The active compounds of the present invention may be administered by any suitable route known to those skilled in the art, preferably in the form of a pharmaceutical composition adapted to such a route, and in a dose effective for the treatment intended. The active compounds and composition may, for example, be administered orally, intravascularly, intraperitoneally, intranasally, intrabronchially, subcutaneously, intramuscularly or topically (including aerosol). For oral administration, the pharmaceutical composition may be in the form of, for example, a tablet, capsule, suspension, or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution, which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound, which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

Formulations for topical use include known gels, creams, oils, and the like. For aerosol delivery, the compounds may be formulated with known aerosol exipients, such as saline, and administered using commercially available nebulizers. Formulation in a fatty acid source may be used to enhance biocompatibility. Aerosol delivery is the preferred method of delivery to the lung for prevention application.

For rectal administration, the active ingredient may be formulated into suppositories using bases, which are solid at room temperature and melt or dissolve at body temperature. Commonly used bases include coca butter, glycerinated gelatin, hydrogenated vegetable oil, polyethylene glycols of various molecular weights, and fatty esters of polyethylene stearate.

Dosages and Dosage Regimen

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex and medical condition of the subject, the severity of the disease, the route and frequency of administration, and the particular compound employed, the location, as well as the pharmacokinetic properties of the individual treated, and thus may vary widely. The dosage will generally be lower if the compounds are administered locally rather than systemically, and for prevention rather than for treatment.

Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain a selective cyclooxygenase-1 inhibitor in the range of about 0.1 to 2000 mg, preferably in the range of about 0.5 to 500 mg and most preferably between about 1 and 200 mg. A daily dose of about 0.01 to 100 mg/kg body weight, preferably between about 0.5 and about 50 mg/kg body weight and most preferably from about 0.1 to 20 mg/kg body weight, may be appropriate. The daily dose can be administered in one, two, three, four, or more doses per day, preferably one or two doses per day.

Combination Therapy

The methods of the present invention may further comprise the administration of other active ingredients or therapies in combination with the administration of the selective cyclooxygenase-1 inhibitor. Such combination therapies include, for example, the administration of other active drugs used in the treatment of cardiovascular-related conditions in combination with the selective cyclooxygenase-1 inhibitor employed in the present methods. The active drugs administered with the selective cyclooxygenase-1 inhibitor can include, for example, the drugs selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, neutral endopeptidase inhibitors (such as omapatrilat), aldosterone receptor antagonists (such as eplerenone and spironolactone), vasodilators, and diuretics. Other active drugs that can be co-administered with a cyclooxygenase-1 inhibitor include, but are not limited to, members of the group consisting of lipid-lowering drugs (including apical sodium bile acid transport inhibitors, cholesterol absorption inhibitors, fibrates, niacin, statins, cholesteryl ester transfer protein inhibitors, and bile acid sequestrants), anti-oxidants (including vitamin E and probucol), and IIIbIIIa antagonists (including xemilofiban and orbofiban).

Angiotensin-II receptor antagonists (“A-II antagonists”) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; and valsartan, which may be prepared as disclosed in U.S. Pat. No. 5,399,578. The disclosures of all such U.S. patents are incorporated herein by reference.

Angiotensin Converting Enzyme Inhibitors (“ACE-Inhibitors”) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361. The disclosures of all such U.S. patents are incorporated herein by reference.

Alpha-adrenergic receptor blockers (alpha- or a-blockers) which are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol, which may be prepared as disclosed above; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may top prepared as disclosed in U.S. Pat. No. 3,669,968; and yohimbine, which may be isolated from natural sources according to methods well known to those skilled in the art. The disclosures of all such U.S. patents are incorporated herein by reference.

Beta-adrenergic receptor blockers (“beta-” or “β-blockers”) which are within the scope of this invention include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. No. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071; buprandolol, which may be prepared as disclosed in U.S. Pat. No. 3,309,406; bubridine hydrochloride, which may be prepared as disclosed in French Patent No. 1,390,056; butofilolol, which may be prepared as disclosed in U.S. Pat. No. 4,252,825; carazolol, which may be prepared as disclosed in German Patent No. 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; cloranolol, which may be prepared as disclosed in German Patent No. 2,213,044; dilevalol, which may be prepared as disclosed in Clifton et al., Journal of Medicinal Chemistry, 1982 25, 670; epanolol, which may be prepared as disclosed in European Patent Publication Application No. 41,491; indenolol, which may be prepared as disclosed in U.S. Pat. No. 4,045,482; labetalol, which may be prepared as disclosed in U.S. Pat. No. 4,012,444; levobunolol, which may be prepared as disclosed in U.S. Pat. No. 4,463,176; mepindolol, which may be prepared as disclosed in Seeman et al., Helv. Chim. Acta, 1971, 54 241; metipranolol, which may be prepared as disclosed in Czechoslovakian Patent Application No. 128,471; metoprolol, which may be prepared as disclosed in U.S. Pat. No. 3,873,600; moprolol, which may be prepared as disclosed in U.S. Pat. No. 3,501,769; nadolol, which may be prepared as disclosed in U.S. Pat. No. 3,935,267; nadoxolol, which may be prepared as disclosed in U.S. Pat. No. 3,819,702; nebivalol, which may be prepared as disclosed in U.S. Pat. No. 4,654,362; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,357; propranolol, which may be prepared as disclosed in U.S. Pat. Nos. 3,337,628 and 3,520,919; sotalol, which may be prepared as disclosed in Uloth et al., Journal of Medicinal Chemistry, 1966 9, 88; sufinalol, which may be prepared as disclosed in German Patent No. 2,728,641; talindol, which may be prepared as disclosed in U.S. Pat. Nos. 3,935,259 and 4,038,313; tertatolol, which may be prepared as disclosed in U.S. Pat. No. 3,960,891; tilisolol, which may be prepared as disclosed in U.S. Pat. No. 4,129,565; timolol, which may be prepared as disclosed in U.S. Pat. No. 3,655,663; toliprolol, which may be prepared as disclosed in U.S. Pat. No. 3,432,545; and xibenolol, which may be prepared as disclosed in U.S. Pat. No. 4,018,824. The disclosures of all such U.S. patents are incorporated herein by reference.

Calcium channel blockers which are within the scope of this invention include, but are not limited to: bepridil, which may be prepared as disclosed in U.S. Pat. No. 3,962, 238 or U.S. Reissue No. 30,577; clentiazem, which may be prepared as disclosed in U.S. Pat. No. 4,567,175; diltiazem, which may be prepared as disclosed in U.S. Pat. No. 3,562,257; fendiline, which may be prepared as disclosed in U.S. Pat. No. 3,262,977; gallopamil, which may be prepared as disclosed in U.S. Pat. No. 3,261,859; mibefradil, which may be prepared as disclosed in U.S. Pat. No. 4,808,605; prenylamine, which may be prepared as disclosed in U.S. Pat. No. 3,152,173; semotiadil, which may be prepared as disclosed in U.S. Pat. No. 4,786,635; terodiline, which may be prepared as disclosed in U.S. Pat. No. 3,371,014; verapamil, which may be prepared as disclosed in U.S. Pat. No. 3,261,859; amlodipine, which may be prepared as disclosed in U.S. Pat. No. 4,572,909; aranipine, which may be prepared as disclosed in U.S. Pat. No. 4,572,909; bamidipine, which may be prepared as disclosed in U.S. Pat. No. 4,220,649; benidipine, which may be prepared as disclosed in European Patent Application Publication No. 106,275; cilnidipine, which may be prepared as disclosed in U.S. Pat. No. 4,672,068; efonidipine, which may be prepared as disclosed in U.S. Pat. No. 4,885,284; elgodipine, which may be prepared as disclosed in U.S. Pat. No. 4,952,592; felodipine, which may be prepared as disclosed in U.S. Pat. No. 4,264,611; isradipine, which may be prepared as disclosed in U.S. Pat. No. 4,466,972; lacidipine, which may be prepared as disclosed in U.S. Pat. No. 4,801,599; lercanidipine, which may be prepared as disclosed in U.S. Pat. No. 4,705,797; manidipine, which may be prepared as disclosed in U.S. Pat. No. 4,892,875; nicardipine, which may be prepared as disclosed in U.S. Pat. No. 3,985,758; nifedipine, which may be prepared as disclosed in U.S. Pat. No. 3,485,847; nilvadipine, which may be prepared as disclosed in U.S. Pat. No. 4,338,322; nimodipine, which may be prepared as disclosed in U.S. Pat. No. 3,799,934; nisoldipine, which may be prepared as disclosed in U.S. Pat. No. 4,154,839; nitrendipine, which may be prepared as disclosed in U.S. Pat. No. 3,799,934; cinnarizine, which may be prepared as disclosed in U.S. Pat. No. 2,882,271; flunarizine, which may be prepared as disclosed in U.S. Pat. No. 3,773,939; lidoflazine, which may be prepared as disclosed in U.S. Pat. No. 3,267,104; lomerizine, which may be prepared as disclosed in U.S. Pat. No. 4,663,325; bencyclane, which may be prepared as disclosed in Hungarian Patent No. 151,865; etafenone, which may be prepared as disclosed in German Patent No. 1,265,758; and perhexiline, which may be prepared as disclosed in British Patent No. 1,025,578. The disclosures of all such U.S. Patents are incorporated herein by reference.

The term “vasodilator”, where used herein, is meant to include cerebral vasodilators, coronary vasodilators and peripheral vasodilators. Cerebral vasodilators within the scope of this invention include, but are not limited to: bencyclane, which may be prepared as disclosed above; cinnarizine, which may be prepared as disclosed above; citicoline, which may be isolated from natural sources as disclosed in Kennedy et al., Journal of the American Chemical Society, 1955, 77 250 or synthesized as disclosed in Kennedy, Journal of Biological Chemistry, 1956, 222 185; cyclandelate, which may be prepared as disclosed in U.S. Pat. No. 3,663,597; ciclonicate, which may be prepared as disclosed in German Patent No. 1,910,481; diisopropylamine dichloroacetate, which may be prepared as disclosed in British Patent No. 862,248; ebumamonine, which may be prepared as disclosed in Hermann et al., Journal of the American Chemical Society, 1979,101, 1540; fasudil, which may be prepared as disclosed in U.S. Pat. No. 4,678,783; fenoxedil, which may be prepared as disclosed in U.S. Pat. No. 3,818,021; flunarizine, which maybe prepared as disclosed in U.S. Pat. No. 3,773,939; ibudilast, which may be prepared as disclosed in U.S. Pat. No. 3,850,941; ifenprodil, which may be prepared as disclosed in U.S. Pat. No. 3,509,164; lomerizine, which may be prepared as disclosed in U.S. Pat. No. 4,663,325; nafronyl, which may be prepared as disclosed in U.S. Pat. No. 3,334,096; nicametate, which may be prepared as disclosed in Blicke et al., Journal of the American Chemical Society, 1942 64 1722; nicergoline, which may be prepared as disclosed above; nimodipine, which may be prepared as disclosed in U.S. Pat. No. 3,799,934; papaverine, which may be prepared as reviewed in Goldberg, Chem. Prod. Chem. News, 1954 17, 371; pentifylline, which may be prepared as disclosed in German Patent No. 860,217; tinofedrine, which may be prepared as disclosed in U.S. Pat. No. 3,563,997; vincamine, which may be prepared as disclosed in U.S. Pat. No. 3,770,724; vinpocetine, which may be prepared as disclosed in U.S. Pat. No. 4,035,750; and viquidil, which may be prepared as disclosed in U.S. Pat. No. 2,500,444. The disclosures of all such U.S. patents are incorporated herein by reference.

Coronary vasodilators within the scope of this invention include, but are not limited to: amotriphene, which may be prepared as disclosed in U.S. Pat. No. 3,010,965; bendazol, which may be prepared as disclosed in J. Chem. Soc. 1958, 2426; benfurodil hemisuccinate, which may be prepared as disclosed in U.S. Pat. No. 3,355,463; benziodarone, which may be prepared as disclosed in U.S. Pat. No. 3,012,042; chloracizine, which may be prepared as disclosed in British Patent No. 740,932; chromonar, which may be prepared as disclosed in U.S. Pat. No. 3,282,938; clobenfural, which may be prepared as disclosed in British Patent No. 1.160,925; clonitrate, which may be prepared from propanediol according to methods well known to those skilled in the art, e.g., see Annalen, 1870,155,165; cloricromen, which may be prepared as disclosed in U.S. Pat. No. 4,452,811; dilazep, which may be prepared as disclosed in U.S. Pat. No. 3,532,685; dipyridamole, which maybe prepared as disclosed in British Patent No. 807,826; droprenilamine, which maybe prepared as disclosed in German Patent No. 2,521,113; efloxate, which may be prepared as disclosed in British Patent Nos. 803,372 and 824,547; erythrityltetranitrate, which may be prepared by nitration of erythritol according to methods well-known to those skilled in the art; etafenone, which may be prepared as disclosed in German Patent No. 1,265,758; fendiline, which may be prepared as disclosed in U.S. Pat. No. 3,262,977; floredil, which may be prepared as disclosed in German Patent No. 2,020,464; ganglefene, which may be prepared as disclosed in U.S.S.R. Patent No. 115,905; hexestrol, which may be prepared as disclosed in U.S. Pat. No. 2,357,985; hexobendine, which may be prepared as disclosed in U.S. Pat. No. 3,267,103; itramin tosylate, which may be prepared as disclosed in Swedish Patent No. 168,308; khellin, which may be prepared as disclosed in Baxter et al., Journal of the Chemical Society, 1949, S30; lidoflaznve, which may be prepared as disclosed in U.S. Pat. No. 3,267,104; mannitol hexanitrate, which may be prepared by the nitration of mannitol according to methods well-known to those skilled in the art; medibazine, which may be prepared as disclosed in U.S. Pat. No. 3,119,826; nitroglycerin; pentaerythritol tetranitrate, which may be prepared by the nitration of pentaerythritol according to methods well-known to those skilled in the art; pentrinitrol, which may be prepared as disclosed in German Patent No. 638,422-3; perhexilline, which may be prepared as disclosed above; pimefylline, which may be prepared as disclosed in U.S. Pat. No. 3,350,400; prenylamine, which may be prepared as disclosed in U.S. Pat. No. 3,152,173; propatyl nitrate, which may be prepared as disclosed in French Patent No. 1,103,113; trapidil, which may be prepared as disclosed in East German Patent No. 55,956; tricromyl, which may be prepared as disclosed in U.S. Pat. No. 2,769,015; trimetazidine, which may be prepared as disclosed in U.S. Pat. No. 3,262,852; trolnitrate phosphate, which maybe prepared by nitration of triethanolamine followed by precipitation with phosphoric acid according to methods well-known to those skilled in the art; visnadine, which may be prepared as disclosed in U.S. Pat. Nos. 2,816,118 and 2,980,699.

The disclosures of all such U.S. patents are incorporated herein by reference. Peripheral vasodilators within the scope of this invention include, but are not limited to: aluminum nicotinate, which may be prepared as disclosed in U.S. Pat. No. 2,970,082; bamethan, which may be prepared as disclosed in Corrigan et al., Journal of the American Chemical Society, 1945, 67 1894; bencyclane, which may be prepared as disclosed above; betahistine, which may be prepared as disclosed in Walter et al.; Journal of the American Chemical Society, 1941, 63, 2771; bradykinin, which may be prepared as disclosed in Hamburg et al., Arch. Biochem. Biophys., 1958, 76 252; brovincamine, which may be prepared as disclosed in U.S. Pat. No. 4,146,643; bufeniode, which may be prepared as disclosed in U.S. Pat. No. 3,542,870; buflomedil, which may be prepared as disclosed in U.S. Pat. No. 3,895,030; butalamine, which may be prepared as disclosed in U.S. Pat. No. 3,338,899; cetiedil, which may be prepared as disclosed in French Patent Nos. 1,460,571; ciclonicate, which may be prepared as disclosed in German Patent No. 1910,481; cinepazide, which may be prepared as disclosed in Belgian Patent No. 730,345; cinnarizine, which may be prepared as disclosed above;

cyclandelate, which may be prepared as disclosed above; diisopropylamine dichloroacetate, which maybe prepared as disclosed above; eledoisin, which may be prepared as disclosed in British Patent No. 984,810; fenoxedil, which may be prepared as disclosed above; flunarizine, which may be prepared as disclosed above; hepronicate, which may be prepared as disclosed in U.S. Pat. No. 3,384,642; ifenprodil, which may be prepared as disclosed above; iloprost, which may be prepared as disclosed in U.S. Pat. No. 4,692,464; inositol niacinate, which may be prepared as disclosed in Badgett et al., Journal of the American Chemical Society, 1947 69, 2907; isoxsuprine, which may be prepared as disclosed in U.S. Pat. No. 3,056,836; kallidin, which may be prepared as disclosed in Biochem. Biophys. Res. Commun.,1961, 6, 210; kallikrein, which may be prepared as disclosed in German Patent No. 1,102,973; moxisylyte, which may be prepared as disclosed in German Patent No. 905,738; nafronyl, which may be prepared as disclosed above; nicametate, which may be prepared as disclosed above; nicergoline, which may be prepared as disclosed above; nicofuranose, which may be prepared as disclosed in Swiss Patent No. 366,523; nylidrin, which may be prepared as disclosed in U.S. Pat. Nos. 2,661,372 and 2,661,373; pentifylline, which may be prepared as disclosed above; pentoxifylline, which may be prepared as disclosed in U.S. Pat. No. 3,422,107; piribedil, which may be prepared as disclosed in U.S. Pat. No. 3,299,067; prostaglandin El, which may be prepared by any of the methods referenced in the Merck Index, Twelfth Edition, Budaveri, Ed., New Jersey, 1996, p. 1353; suloctidil, which may be prepared as disclosed in German Patent No. 2,334,404; tolazoline, which may be prepared as disclosed in U.S. Pat. No. 2,161,938; and xanthinolniacinate, which may be prepared as disclosed in German Patent No. 1,102,750 or Korbonits et al., Acta. Pharm. Hung., 1968, 38, 98. The disclosures of all such U.S. patents are incorporated herein by reference.

The term “diuretic”, within the scope of this invention, includes, but is not limited to, diuretic benzothiadiazine derivatives, diuretic organomercurials, diuretic purines, diuretic steroids (including diuretic steroids having no substantial activity as an aldosterone receptor antagonist), diuretic sulfonamide derivatives, diuretic uracils and other diuretics such as amanozine, which may be prepared as disclosed in Austrian Patent No. 168,063; amiloride, which may be prepared as disclosed in Belgian Patent No. 639,386; arbutin, which may be prepared as disclosed in Tschitschibabin, Annalen,1930, 478, 303; chlorazanil, which may be prepared as disclosed in Austrian Patent No. 168,063; ethacrynic acid, which may be prepared as disclosed in U.S. Pat. No. 3,255,241; etozolin, which may be prepared as disclosed in U.S. Pat. No. 3,072,653; hydracarbazine, which may be prepared as disclosed in British Patent No. 856,409; isosorbide, which may be prepared as disclosed in U.S. Pat. No. 3,160,641; mannitol; metochalcone, which may be prepared as disclosed in Freudenberg et al., Ber., 1957, 90, 957; muzolimine, which may be prepared as disclosed in U.S. Pat. No. 4,018,890; perhexiline, which may be prepared as disclosed above; ticrynafen, which may be prepared as disclosed in U.S. Pat. No. 3,758,506; triamterene which may be prepared as disclosed in U.S. Pat. No. 3,081,230; and urea. The disclosures of all such U.S. patents are incorporated herein by reference.

Diuretic benzothiadiazine derivatives within the scope of this invention include, but are not limited to: althiazide, which may be prepared as disclosed in British Patent No. 902,658; bendroflumethiazide, which may be prepared as disclosed in U.S. Pat. No. 3,265,573; benzthiazide, McManus et al., 136th Am. Soc. Meeting (Atlantic City, September 1959), Abstract of papers, pp 13-0; benzylhydrochlorothiazide, which may be prepared as disclosed in U.S. Pat. No. 3,108,097; buthiazide, which may be prepared as disclosed in British Patent Nos. 861,367 and 885,078; chlorothiazide, which may be prepared as disclosed in U.S. Pat. Nos. 2,809,194 and 2,937,169; chlorthalidone, which may be prepared as disclosed in U.S. Pat. No. 3,055,904; cyclopenthiazide, which may be prepared as disclosed in Belgian Patent No. 587,225; cyclothiaide, which may be prepared as disclosed in Whitehead et al., Journal of Organic Chemistry, 1961,26, 2814; epithiazide, which may be prepared as disclosed in U.S. Pat. No. 3,009,911; ethiazide, which may be prepared as disclosed in British Patent No. 861,367; fenquizone, which may be prepared as disclosed in U.S. Pat. No. 3,870,720; indapamide, which may be prepared as disclosed in U.S. Pat. No. 3,565,911; hydrochlorothiazide, which may be prepared as disclosed in U.S. Pat. No. 3,164,588; hydroflumethiazide, which may be prepared as disclosed in U.S. Pat. No. 3,254,076; methyclothiazide, which may be prepared as disclosed in Close et al., Journal of the American Chemical Society, 1960, 82,1132; meticrane, which may be prepared as disclosed in French Patent Nos. M2790 and 1,365,504; metolazone, which may be prepared as disclosed in U.S. Pat. No. 3,360,518; paraflutizide, which may be prepared as disclosed in Belgian Patent No. 620,829; polythiazide, which may be prepared as disclosed in U.S. Pat. No. 3,009,911; quinethazone, which may be prepared as disclosed in U.S. Pat. No. 2,976,289; teclothiazide, which may be prepared as disclosed in Close et al., Journal of the American Chemical Society, 1960, 82, 1132; and trichlormethiazide, which may be prepared as disclosed in deStevens et al., Experientia, 1960,16, 113. The disclosures of all such U.S. patents are incorporated herein by reference.

Diuretic sulfonamide derivatives within the scope of this invention include, but are not limited to: acetazolamide, which may be prepared as disclosed in U.S. Pat. No. 2,980,679; ambuside, which may be prepared as disclosed in U.S. Pat. No. 3,188,329; azosernide, which may be prepared as disclosed in U.S. Pat. No. 3,665,002; bumetamide, which may be prepared as disclosed in U.S. Pat. No. 3,634,583; butazolamide, which may be prepared as disclosed in British Patent No. 769,757; chloraminophenamide, which may be prepared as disclosed in U.S. Pat. Nos. 2,809,194, 2,965,655 and 2,965,656; clofenamide, which may be prepared disclosed in Olivier, Rec. Trav. Chim., 1918, 37 307; clopamide, which may be prepared as disclosed in U.S. Pat. No. 3,459,756; clorexolone, which may be prepared as disclosed in U.S. Pat. No. 3,183,243; disulfamide, which may be prepared as disclosed in British Patent No. 851,287; ethoxolamide, which may be prepared as disclosed in British Patent No. 795,174; furosemide, which may be prepared as disclosed in U.S. Pat. No. 3,058,882; mefruside, which may be prepared as disclosed in U.S. Pat. No. 3,356,692; methazolamide, which may be prepared as disclosed in U.S. Pat. No. 2,783,241; piretamide, which may be prepared as disclosed in U.S. Pat. No. 4,010,273; torasemide, which may be prepared as disclosed in U.S. Pat. No. 4,018,929; tripamide, which may be prepared as disclosed in Japanese Patent No. 73 05,585; and xipamide, which may be prepared as disclosed in U.S. Pat. No. 3,567,777. The disclosures of all such U.S. patents are incorporated herein by reference.

Statins within the scope of this invention include, but are not limited to: atorvastatin, which may be prepared as disclosed in EP 409,281; fluvastatin, which may be prepared as disclosed in EP 244,364; lovastatin, which may be prepared as disclosed in EP 22, 478; mevastatin, which may be prepared as disclosed in JP 56,051,992; pitavastatin, which may be prepared as disclosed in Takano, et al., Tetahedron: Assymetry, (1993), 4(2), 201-4; pravastatin, which may be prepared as disclosed in DE 3,122,499; rosuvastatin, which may be prepared as disclosed in EP 521,471; and simvastatin, which may be prepared as disclosed in EP 33,538.

In one embodiment the selective cyclooxygenase-1 inhibitor can be administered in combination with a renin inhibitor.

In another embodiment the selective cyclooxygenase-1 inhibitor can be administered in combination with an angiotension I antagonist.

In another embodiment the selective cyclooxygenase-1 inhibitor can be administered in combination with an antiotension II antagonist.

In another embodiment the selective cyclooxygenase-1 inhibitor can be administered in combination with an angiotension converting enzyme inhibitor.

In another embodiment the selective cyclooxygenase-1 inhibitor can be administered in combination with an alpha-adrenergic receptor blocker.

In another embodiment the selective cyclooxygenase-1 inhibitor can be administered in combination with a beta-adrenergic receptor blocker. In another embodiment the selective cyclooxygenase-1 inhibitor can be administered in combination with a calcium channel blocker.

In another embodiment the selective cyclooxygenase-1 inhibitor can be administered in combination with a neutral endopeptidase inhibitor (such as omapatrilat).

In another embodiment the selective cyclooxygenase-1 inhibitor can be administered in combination with an aldosterone receptor antagonist (such as eplerenone and spironolactone).

In another embodiment the selective cyclooxygenase-1 inhibitor can be administered in combination with a vasodilator.

In another embodiment the selective cyclooxygenase-1 inhibitor can be administered in combination with a diuretic.

In another embodiment the selective cyclooxygenase-1 inhibitor can be administered in combination with a statin (such as mevastatin, lovastatin, simvastatin, pravastatin, fluvastatin, atorvastatin, rosuvastatin, and pitavastatin).

In another embodiment the selective cyclooxygenase-1 inhibitor can be administered in combination with anti-oxidants (including vitamin E and probucol).

In another embodiment the selective cyclooxygenase-1 inhibitor can be administered in combination with a IIbIIIa antagonist (including xemilofiban and orbofiban).

In one embodiment of the invention, the selective COX-1 inhibitor and the drug used for treating or preventing cardiovascular-related conditions may be administered substantially simultaneously, meaning that both agents may be provided in a single dosage, for example by mixing the agents and incorporating the mixture into a single capsule. Alternatively, the selective COX-1 inhibitor and drug used for treating or preventing cardiovascular-related conditions may be administered substantially simultaneously by administration in separate dosages within a short time period, for example within 5 minutes or less. Alternatively, the selective COX-1 inhibitor and drug used for treating or preventing cardiovascular-related conditions may be administered sequentially, meaning that separate dosages, and possibly even separate dosage forms of the selective COX-1 inhibitor and drug used for treating or preventing cardiovascular-related conditions may be administered at separate times, for example on a staggered schedule but with equal frequency of administration of the selective COX-1 inhibitor and drug used for treating or preventing cardiovascular-related conditions. Of course, it is also possible that the selective COX-1 inhibitor may be administered either more or less frequently than the drug used for treating or preventing cardiovascular-related conditions.

Administration of a selective cyclooxygenase-1 inhibitor also can be effected in combination with one or more of non-drug therapies, such as non-drug therapies associated with the treatment of restenosis. For example, conventional treatment of restenosis resulting from angioplasty includes therapies such as exposing the artery at the site of injury to a source of radiation to inhibit restrictive neointima growth and inserting an endolumenal stent at the site of angioplasty.

In one embodiment, the selective cyclooxygenase-1 inhibitor can be administered in combination with exposure of an angioplastied artery at the site of injury to a source of radiation to inhibit restrictive neointima growth. Although radiation monotherapy has been used to prevent restenosis after angioplasty, Powers et al., Int. J. Radiat. Oncol. Biol, Vol. 45(3), pp. 753-59 (Oct. 1, 1999), report findings in a study involving a canine model that indicate that adventitial fibrosis increases with increasing dose of radiation and can contribute to adverse late vascular remodeling. The proposed combination therapy would permit the use of dosages of radiation below conventional monotherapeutic dosages of radiation and would result in fewer side-effects or adverse effects relative to such radiation monotherapy.

In another embodiment, the stent itself comprises the selective cyclooxygenase-1 inhibitor and is used as a carrier to effect local delivery of the selective cyclooxygenase-1 inhibitor to the injured vessel. The selective cyclooxygenase-1 inhibitor is coated on, adsorbed on, affixed to or present on the surface of the stent or is otherwise present in or on the matrix of the stent, either alone or in combination with other active drugs and pharmaceutically acceptable carriers, adjuvants, binding agents and the like. The stent preferably comprises the selective cyclooxygenase-1 inhibitor in the form of an extended release composition that provides for release of the antagonist over an extended period of time.

Kits

The present invention further comprises kits that are suitable for use in performing the methods of treatment or prevention of an inflammation-related cardiovascular condition as described above. In one embodiment, the kit contains a first dosage form comprising a selective cyclooxygenase-1 inhibitor and a second dosage form comprising a drug used in the treatment or prevention of a cardiovascular condition, in quantities sufficient to carry out the methods of the present invention. Preferably, the first dosage form and the second dosage form together comprise a therapeutically effective amount for the prevention and/or treatment of inflammation-related cardiovascular conditions.

In one embodiment, the cyclooxygenase-1 selective inhibitor is a compound of Formula 1, and the drug used in the treatment or prevention of a cardiovascular condition is selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, neutral endopeptidase inhibitors, aldosterone receptor antagonists, vasodilators, diuretics, lipid-lowering drugs, anti-oxidants, and IIbIIIa antagonists, as described above, in quantities sufficient to carry out the methods of the present invention. In one embodiment, the drug used in the treatment or prevention of a cardiovascular condition is a calcium channel blocker selected from the group consisting of amlodipine, nifedipine, and verapamil. In another embodiment, the drug used in the treatment or prevention of a cardiovascular condition is a statin selected from the group consisting of mevastatin, lovastatin, simvastatin, pravastatin, fluvastatin, atorvastatin, rosuvastatin, and pitavastatin. In a preferred embodiment, the statin is selected from the group consisting of atorvastatin, simvastatin, pravastatin, and rosuvastatin. In another preferred embodiment, the statin is atorvastatin.

In another embodiment, the cyclooxygenase-1 selective inhibitor is selected from the group identified in Table 1, and the drug used in the treatment or prevention of a cardiovascular condition is selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, neutral endopeptidase inhibitors, aldosterone receptor antagonists, vasodilators, diuretics, lipid-lowering drugs, anti-oxidants, and IIbIIIa antagonists, as described above, in quantities sufficient to carry out the methods of the present invention. In one embodiment, the drug used in the treatment or prevention of a cardiovascular condition is a calcium channel blocker selected from the group consisting of amlodipine, nifedipine, and verapamil. In another embodiment, the drug used in the treatment or prevention of a cardiovascular condition is a statin selected from the group consisting of mevastatin, lovastatin, simvastatin, pravastatin, fluvastatin, atorvastatin, rosuvastatin, and pitavastatin. In a preferred embodiment, the statin is selected from the group consisting of atorvastatin, simvastatin, pravastatin, and rosuvastatin. In another preferred embodiment, the statin is atorvastatin.

The following examples contain detailed descriptions of the method of the present invention. These detailed descriptions fall within the scope of, and serve to exemplify, the invention. These detailed descriptions are presented for illustrative purposes only and are not intended as a restriction on the scope of the invention.

### Example 1

### Synthesis of 1,5-diphenyl-3-(trifluoromethyl)pyrazole

The title compound was prepared from acetophenone and phenylhydrazine hydrochloride using the procedure described in Journal of Medicinal Chemistry, vol. 40, pages 1347-65 (Penning, et al., incorporated herein by reference), in 36% yield: ¹H NMR (acetone-d₆) ^{δ}7.00 (s, 1H), 7.31-7.47 (m, 10H); ¹⁹F NMR (acetone-d₆) ^{δ}−6-63.10 (s).

### Example 2

### Synthesis of 1,5-bis(4-chlorophenyl)-3-(trifluoromethyl)pyrazole

The title compound was prepared from 4′-chloroacetophenone and (4-chlorophenyl)hydrazine hydrochloride using the procedure described in Journal of Medicinal Chemistry, vol. 40, pages 1347-65 (Penning, et al., incorporated herein by reference), in 49% yield: ¹H NMR (acetone-d₆) ^{δ}7.06 (s, 1H), 7.36-7.54 (m, 8H); ¹⁹F NMR (acetone-d₆) ^{δ}−63.22 (s).

### Example 3

### Synthesis of 1,5-bis(4-methoxyphenyl)-3-(trifluoromethyl)pyrazole

The title compound was prepared from 4′-methoxyacetophenone and (4-methoxyphenyl)hydrazine hydrochloride using the procedure described in Journal of Medicinal Chemistry, vol. 40, pages 1347-65 (Penning, et al., incorporated herein by reference), in 76% yield: ¹H NMR (acetone-d₆) ^{δ}3.81 (d, J=1.4 Hz, 3H), 3.85 (d, J=1.4 Hz, 3H), 6.88 (s, 1H), 6.94 (dd, J=8.9, 1.2 Hz, 2H), 7.01 (dd, J=8.9, 1.2 Hz, 2H), 7.23 (dd, J=8.9, 1.2 Hz, 2H), 7.27 (dd, J=8.9, 1.4 Hz, 2H); ¹⁹F NMR (acetone-d₆) ^{δ}−63.02 (s).

### Example 4

### Synthesis of 1-(4-chlorophenyl)-5-(4-methoxyphenyl)-3-(trifluoromethyl)pyrazole

The title compound was prepared from 4′-methoxyacetophenone and (4-chlorophenyl)hydrazine hydrochloride using the procedure described in Journal of Medicinal Chemistry, vol.40, pages 1347-65 (Penning, et al., incorporated herein by reference), in 48% yield: ¹H NMR (CDCl₃) ^{δ}3.82 (s, 3H), 6.65 (s, 1H), 6.87 (d, J=8.7 Hz, 2H), 7.13 (d, J=8.7 Hz, 2H), 7.25 (d, J=8.7 Hz, 2H), 7.33 (d, J=8.7 Hz, 2H).

### Example 5

### Synthesis of 5-(4-Chlorophenyl)-1-(4-methoxyphenyl)-3-(trifluoromethyl)pyrazole

The title compound was prepared from 4′-chloroacetophenone and (4-methoxyphenyl)hydrazine hydrochloride using the procedure described in Journal of Medicinal Chemistry, vol. 40, pages 1347-65 (Penning, et al., incorporated herein by reference), in 72% yield: ¹H NMR (CDCl₃) δ 3.82 (s, 3H), 6.72 (s, 1H), 6.87 (d, J=8.6 Hz, 2H), 7.14 (d, J=8.6 Hz, 2H), 7.21 (d, J=9.0 Hz, 2H), 7.29 (d, J=8.6 Hz, 2H); ¹⁹F NMR (CDCl₃) ^{δ}−63.02.

### Example 6

### Effect of Cyclooxygenase Inhibition on the Development of Atherosclerosis in Cholesterl-Fed, apoE Knockout Mice

The role of cyclooxygenase-1 and cyclooxygenase-2 on the development of atherosclerosis in apoE knockout mice fed a 1% cholesterol diet was evaluated. Although COX-2 is expressed in inflammatory and vascular smooth muscle cells found in human atherosclerotic plaques, the results of rat studies indicate that COX-1 is responsible for the intimal hyperplasia following balloon injury of the carotid artery. In the studies, animals were randomised to receive vehicle alone (n=20), the selective COX-1 inhibitor (5-(4-Chlorophenyl)-1-(4-methoxyphenyl)-3-(trifluoromethyl)pyrazole, Example 5) at a daily dose of 15 mg/kg (n=20) or the selective COX-2 inhibitor (4-[5-(4-chlorophenyl)-3-(trifluoromethyl)-1H-pyrazol -1-yl]benzenesulfonamide) at 6 mg/kg (n=20). ApoE^{-/-} fed a normal chow (n=20) and wildtype littermates (n=40) were used as controls for the study. Twenty-four hour urine samples were collected and the aortae harvested following 10 weeks of treatment.

Immunohistochemistry showed an increase in COX-1 and COX-2 staining in all apoE^{-/-} animals and localised the increased expression to atherosclerotic lesions. COX-2 dependent PGE₂ generation was also increased in the vessels of ApoE^{-/-} animals fed a 1% diet compared with controls that was (5.6±0.88 pg/ml vs. 2.7±0.64 pg/ml, p<0.05). Urinary TXB₂ excretion was elevated in apoE^{-/-} animals fed a 1% cholesterol compared with wildtype controls (20.7±4.02 ng/ml vs. 3.75±1.23 ng/ml, p<0.05) and this was reduced by the COX-1 inhibitor (2.63±0.59 ng/ml, p<0.05). The COX-1 inhibitor also reduced the extent of lesion development when compared to apoE^{-/-} animals fed a 1% cholesterol diet that received vehicle alone (4.6±0.4% vs. 25.8±4.5%, p<0.01). In contrast, the selective COX-2 inhibitor had no effect on TXB₂ excretion or lesion development (25.8±4.5% vs. 27.9±1.8%). The lesions in animals fed a 1% cholesterol diet showed marked staining for CD41, demonstrating platelet deposition and this was markedly attenuated by 5-(4-chlorophenyl)-1-(4-methoxyphenyl)-3-(trifluoromethyl)pyrazole. Selective inhibition of COX-2 had no effect on CD41, and specifically did not increase platelet deposition. These results indicate that selective inhibition of COX-1 delays the development of atherosclerosis in apoE knockout mice.

### Example 7

### Evaluation of COX-1 and COX-2 Activity in vitro

The methods set forth in this example can be used to evaluate a compound for COX-1 and/or COX-2 inhibition activity in vitro:

A. Preparation of Recombinant Cyclooxygenase Haculoviruses

Recombinant COX-1 and COX-2 were prepared as described by Gierse et al., (J. Biochem., 305, 479-84 (1995)). A 2.0 kb fragment containing the coding region of either human or murine COX-1 or human or murine COX-2 was cloned into BamH1 site of the baculovirus transfer vector pVL1393 (Invitrogen) to generate the baculovirus transfer vectors for COX-1 and COX-2 in a manner similar to the method of D. R. O'Reilly et al. (Baculovirus Expression Vectors: A Laboratory Manual (1992)). Recombinant baculoviruses were isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Cultures Procedures, Texas Agric. Exp.

Station Bull. 1555 (1987). Recombinant viruses were purified by three rounds of plaque purification and high titer (10⁷-10⁸ pfu/mL) stocks of virus were prepared. For large scale production, SF9 insect cells were infected in 10 liter fermentors (0.5×10⁶/mL) with the recombinant baculovirus stock such that the multiplicity of infection was 0.1. After 72 hours the cells were centrifuged and the cell pellet homogenized in Tris/Sucrose (50 mM: 25%, pH 8.0) containing 1% 3-[(3-cholamidopropyl)dimethylammonio]-1-propanesulfonate (CHAPS). The homogenate was centrifuged at 10,000×G for 30 minutes, and the resultant supernatant was stored at −80° C. before being assayed for COX activity.

B. Assay for COX-1 and COX-2 $activity

COX activity was assayed as PGE₂ formed/μg protein/time using an ELISA to detect the prostaglandin released. CHAPS-solubilized insect cell membranes containing the appropriate COX enzyme were incubated in a potassium phosphate buffer (50 mM, pH 8.0) containing epinephrine, phenol, and heme with the addition of arachidonic acid (10 μM). Compounds were pre-incubated with the enzyme for 10-20 minutes prior to the addition of arachidonic acid. Any reaction between the arachidonic acid and the enzyme was stopped after ten minutes at 37° C./room temperature by transferring 40 μl of reaction mix into 160 μl ELISA buffer and 25 μM indomethacin. The PGE₂ formed was measured by standard ELISA technology (Cayman Chemical).

C. Fast assay for COX-1 and COX-2 Activity

COX activity was assayed as PGE₂ formed/μg protein/time using an ELISA to detect the prostaglandin released. CHAPS-solubilized insect cell membranes containing the appropriate COX enzyme were incubated in a potassium phosphate buffer (0.05 M Potassium phosphate, pH 7.5, 2 μM phenol, 1 μM arachidonic acid (10 μM). Compounds were pre-incubated with the enzyme for 10 minutes at 25° C. prior to the addition of arachidonic acid. Any reaction between the arachidonic acid and the enzyme was stopped after two minutes at 37° C./room temperature by transferring 40 μl of reaction mix into 160 μl ELISA buffer and 25 μM indomethacin. The PGE₂ formed was measured by standard ELISA technology (Cayman Chemical).

All mentioned references are incorporated by reference as if here written. When introducing elements of the present invention or the preferred embodiment(s) thereof, the articles “a”, “an”, “the” and “said” are intended to mean that there are one or more of the elements. The terms “comprising”, “including”, and “having” are intended to be inclusive and mean that there may be additional elements other than the listed elements.

Although this invention has been described with respect to specific embodiments, the details of these embodiments are not to be construed as limitations.

What is claimed is:

1. A method for treating or preventing an inflammation-related cardiovascular condition in a subject in need of such prevention, the method comprising administering to the subject a therapeutically effective amount of a selective cyclooxygenase-1 inhibiting compound or a pharmaceutically acceptable salt, tautomer, or prodrug thereof.

2. The method of claim 1 wherein the selective cyclooxygenase-1 inhibiting compound is a compound of Formula I: 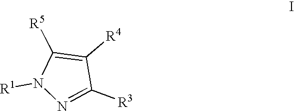
wherein:
R¹ is substituted or unsubstituted aryl;
one of R³ and R⁴ is hydrogen, and the other of R³ and R⁴ is an electron withdrawing group or a substituted or unsubstituted alkyl group; and
R⁵ is substituted or unsubstituted aryl;
or R³ is an electron withdrawing group or a substituted or unsubstituted alkyl group, and R⁴ and R⁵ combine to form a fused ring substituent;
or a pharmaceutically acceptable salt, tautomer or prodrug thereof.

3. The method of claim 2 wherein:
R¹ is substituted or unsubstituted phenyl;
one of R³ and R⁴ is hydrogen, and the other of R³ and R⁴ is selected from the group consisting of halogen, cyano, hydroxy, carboxy, haloalkyl, hydroxyalkyl, and alkoxycarbonyl; and
R⁵ is substituted or unsubstituted phenyl;
or a pharmaceutically acceptable salt, tautomer or prodrug thereof.

4. The method of claim 2 wherein:
R¹ is phenyl, wherein said phenyl is unsubstituted or is substituted with one or more groups selected from the group consisting of halogen, alkyl, haloalkyl, alkoxy and alkylthio;
one of R³ and R⁴ is hydrogen, and the other of R³ and R⁴ is selected from the group consisting of halogen, cyano, hydroxy, carboxy, haloalkyl, hydroxyalkyl, and alkoxycarbonyl; and
R⁵ is phenyl, wherein said phenyl is unsubstituted or is substituted with one or more groups selected from the group consisting of halogen, alkyl, haloalkyl, alkoxy and alkylthio;
or a pharmaceutically acceptable salt, tautomer or prodrug thereof.

5. The method of claim 2 wherein:
R¹ is phenyl, wherein said phenyl is unsubstituted or is substituted with one or more groups selected from the group consisting of halogen, (C₁-C₆)alkyl, halo-(C₁-C₆)alkyl, (C₁-C₆)alkoxy, and (C₁-C₆)alkylthio;
one of R³ and R⁴ is hydrogen, and the other of R³ and R⁴ is selected from the group consisting of halogen, cyano, hydroxy, halo-(C₁-C₆)alkyl, hydroxy-(C₁-C₆)alkyl, and (C₁-C₆)alkoxycarbonyl; and
R⁵ is phenyl, wherein said phenyl is unsubstituted or is substituted with one or more groups selected from the group consisting of halogen, (C₁-C₆)alkyl, halo-(C₁-C₆)alkyl, (C₁-C₆)alkoxy, and (C₁-C₆)alkylthio;
or a pharmaceutically acceptable salt, tautomer or prodrug thereof.

6. The method of claim 2 wherein:
R¹ is phenyl, wherein said phenyl is unsubstituted or is substituted with one or more groups selected from the group consisting of halogen, (C₁-C₂)alkyl, halo-(C₁-C₂)alkyl, (C₁-C₂)alkoxy, and (C₁-C₂)alkylthio;
one of R³ and R⁴ is hydrogen, and the other of R³ and R⁴ is selected from the group consisting of halogen, cyano, hydroxy, halo-(C₁-C₂)alkyl, hydroxy-(C₁-C₂)alkyl, and (C₁-C₂)alkoxycarbonyl; and
R⁵ is phenyl, wherein said phenyl is unsubstituted or is substituted with one or more groups selected from the group consisting of halogen, (C₁-C₂)alkyl, halo-(C₁-C₂)alkyl, (C₁-C₂)alkoxy, and (C₁-C₂)alkylthio;
or a pharmaceutically acceptable salt, tautomer or prodrug thereof.

7. The method of claim 2 wherein one or both of R¹ and R⁵ are phenyl substituents independently mono-substituted at the para position, and the R³ substituent is hydrogen.

8. The method of claim 2 wherein one or both of R¹ and R⁵ are phenyl substituents independently mono-substituted at the para position, and the R⁴ substituent is hydrogen.

9. The method of claim 2 wherein the compound is 1,5-bis(4-methoxyphenyl)-3-(trifluoromethyl)pyrazole, or the pharmaceutically-acceptable salts, tautomers and prodrugs thereof.

10. The method of claim 2 wherein the compound is a 3-(trifluoromethyl)-1,5-diarylpyrazole, or a pharmaceutically-acceptable salt, tautomer or prodrug thereof.

11. The method of claim 10 wherein the compound does not comprise a sulfonamide moiety.

12. The method of claim 2 wherein the compound is a 3-(difluoromethyl)-1,5-diarylpyrazole, or a pharmaceutically acceptable salt, tautomer, or prodrug thereof.

13. The method of claim 12 wherein the 3-(difluoromethyl)-1,5-diarylpyrazole does not comprise a sulfonamide moiety.

14. The method of claim 2 wherein the compound is selected from the group consisting of formulas: 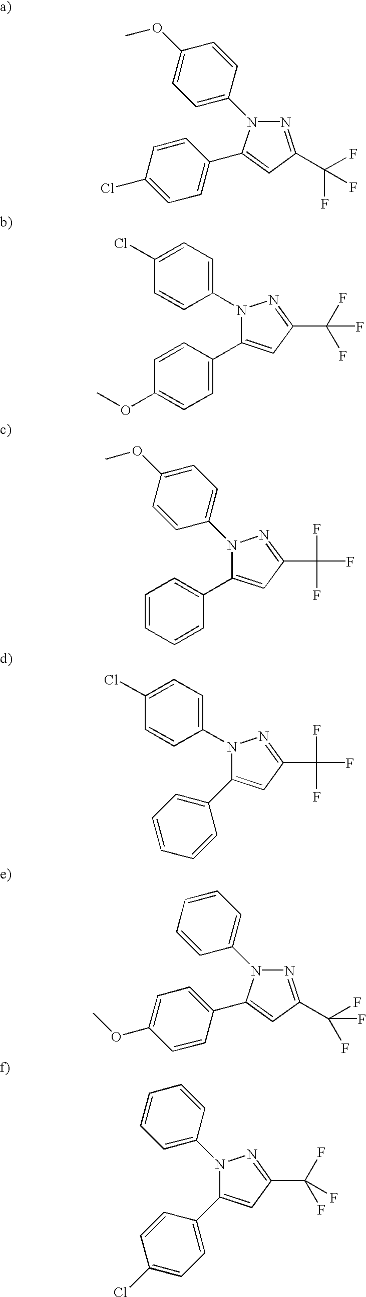 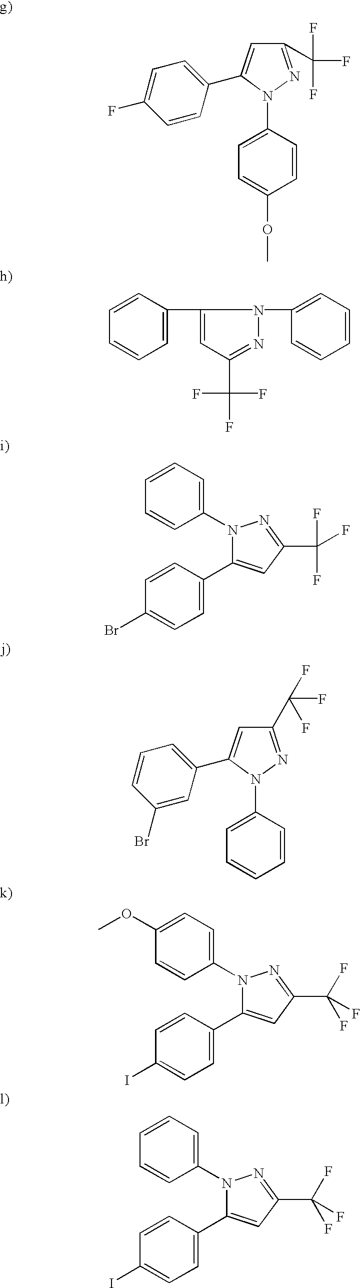 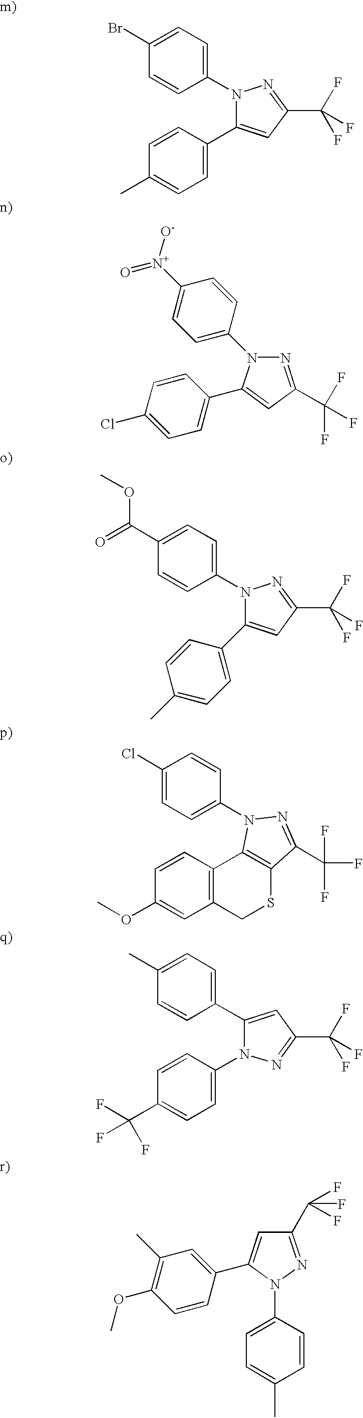 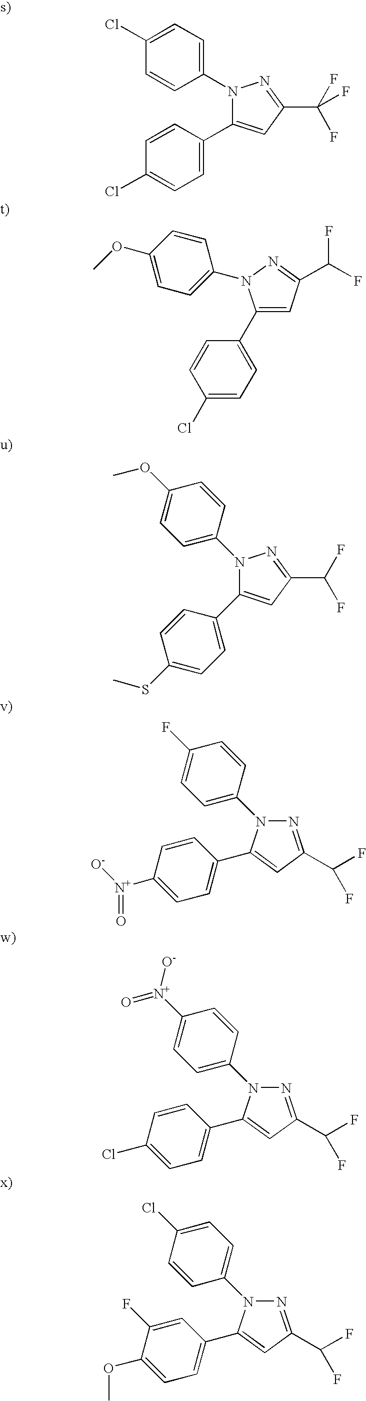 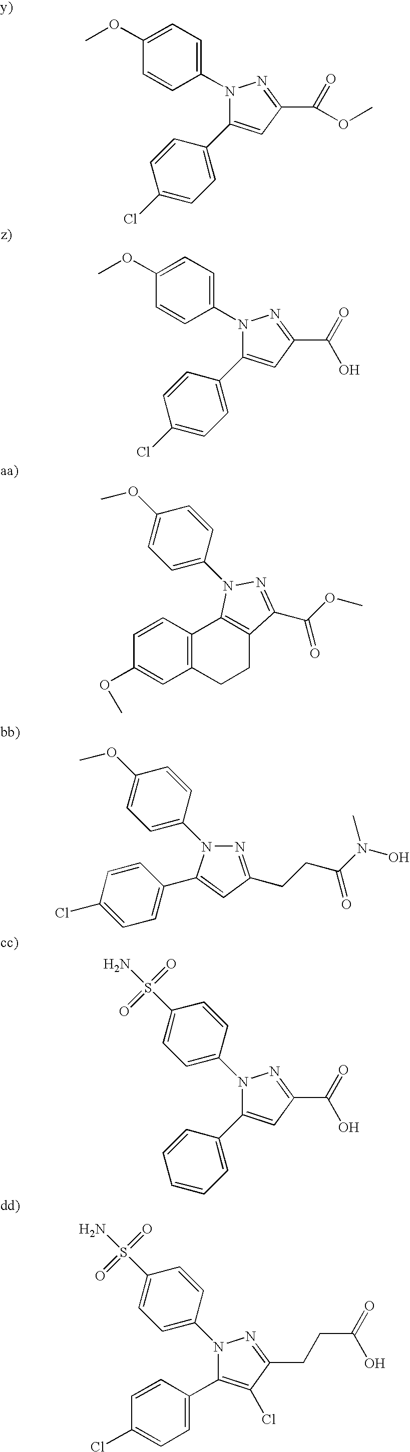 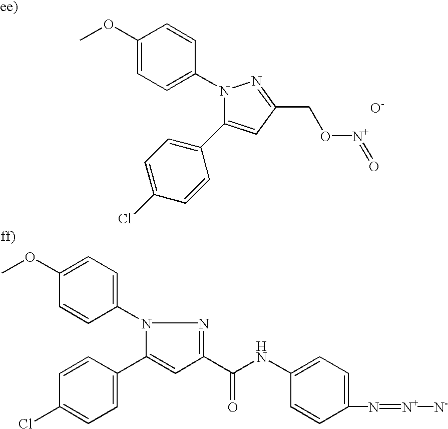
or pharmaceutically acceptable salts, tautomers, or prodrugs thereof.

15. The method of claim 1 wherein the cardiovascular condition is selected from the group consisting of coronary artery disease, aneurysm, arteriosclerosis, atherosclerosis including cardiac transplant atherosclerosis, myocardial infarction, aortic stenosis, renal dysfunction, renal failure, microalbuminuria, heart failure, embolism, stroke, thrombosis (including venous thrombosis), angina (including unstable angina), myocarditis, pericarditis, coronary plaque inflammation, bacterial-induced inflammation (including Chlamydia-induced inflammation), viral induced inflammation, auto-immune-induced inflammation, and inflammation associated with surgical procedures such as vascular grafting including coronary artery bypass surgery, revascularization procedures including angioplasty, stent placement, endarterectomy, and other invasive procedures involving arteries, veins and capillaries.

16. The method of claim 15 wherein the cardiovascular condition is atherosclerosis.

17. The method of claim 15 wherein the cardiovascular condition is thrombosis.

18. The method of claim 15 wherein the cardiovascular condition is restenosis.

19. The method of claim 15 wherein the cardiovascular condition is inflammation or hyperplasia resulting from vascular injury in a subject.

20. The method of claim 1 wherein the cardiovascular condition is caused by or aggravated by cyclooxygenase-1 activity.

21. The method of claim 1 wherein the subject is a mammal.

22. The method of claim 21 wherein the mammal is a human.

23. The method of claim 22 wherein the human is at risk for developing an inflammation-related cardiovascular condition or has previously been diagnosed with an inflammation-related cardiovascular condition.

24. The method claims 1 wherein the cyclooxygenase-1 selective inhibitor is administered to the subject in an amount of about 0.01 to about 100 mg/kg body weight per day.

25. The method of claim 24 wherein the the cyclooxygenase-1 selective inhibitor is administered to the subject in an amount of about 0.5 to about 50 mg/kg body weight per day.

26. The method of claim 25 wherein the the cyclooxygenase-1 selective inhibitor is administered to the subject in an amount of about 0.1 to about 20 mg/kg body weight per day.

27. A method for treating or preventing an inflammation-related cardiovascular condition in a subject in need of such prevention, the method comprising administering to the subject a selective cyclooxygenase-1 inhibiting compound or a pharmaceutically acceptable salt, tautomer, or prodrug thereof in combination with either a drug used in the treatment or prevention of a cardiovascular condition or a non-drug therapy used in the treatment of a cardiovascular condition, such that the combination therapy is therapeutically effective in the treatment or prevention of the inflammation-related cardiovascular condition.

28. The method of claim 27 wherein the selective cyclooxygenase-1 inhibiting compound is a compound of Formula I: 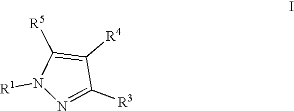
wherein:
R¹ is substituted or unsubstituted aryl;
one of R³ and R⁴ is hydrogen, and the other of R³ and R⁴ is an electron withdrawing group or a substituted or unsubstituted alkyl group; and
R⁵ is substituted or unsubstituted aryl;
or R³ is an electron withdrawing group or a substituted or unsubstituted alkyl group, and R⁴ and R⁵ combine to form a fused ring substituent;
or a pharmaceutically acceptable salt, tautomer or prodrug thereof.

29. The method of claim 28 wherein:
R¹ is substituted or unsubstituted phenyl;
one of R³ and R⁴ is hydrogen, and the other of R³ and R⁴ is selected from the group consisting of halogen, cyano, hydroxy, carboxy, haloalkyl, hydroxyalkyl, and alkoxycarbonyl; and
R⁵ is substituted or unsubstituted phenyl;
or a pharmaceutically acceptable salt, tautomer or prodrug thereof.

30. The method of claim 28 wherein:
R¹ is phenyl, wherein said phenyl is unsubstituted or is substituted with one or more groups selected from the group consisting of halogen, alkyl, haloalkyl, alkoxy and alkylthio;
one of R³ and R⁴ is hydrogen, and the other of R³ and R⁴ is selected from the group consisting of halogen, cyano, hydroxy, carboxy, haloalkyl, hydroxyalkyl, and alkoxycarbonyl; and
R⁵ is phenyl, wherein said phenyl is unsubstituted or is substituted with one or more groups selected from the group consisting of halogen, alkyl, haloalkyl, alkoxy and alkylthio;
or a pharmaceutically acceptable salt, tautomer or prodrug thereof.

31. The method of claim 28 wherein:
R¹ is phenyl, wherein said phenyl is unsubstituted or is substituted with one or more groups selected from the group consisting of halogen, (C₁-C₆)alkyl, halo-(C₁-C₆)alkyl, (C₁-C₆)alkoxy, and (C₁-C₆)alkylthio;
one of R³ and R⁴ is hydrogen, and the other of R³ and R⁴ is selected from the group consisting of halogen, cyano, hydroxy, halo-(C₁-C₆)alkyl, hydroxy-(C₁-C₆)alkyl, and (C₁-C₆)alkoxycarbonyl; and
R⁵ is phenyl, wherein said phenyl is unsubstituted or is substituted with one or more groups selected from the group consisting of halogen, (C₁-C₆)alkyl, halo-(C₁-C₆)alkyl, (C₁-C₆)alkoxy, and (C₁-C₆)alkylthio;
or a pharmaceutically acceptable salt, tautomer or prodrug thereof.

32. The method of claim 28 wherein:
R¹ is phenyl, wherein said phenyl is unsubstituted or is substituted with one or more groups selected from the group consisting of halogen, (C₁-C₂)alkyl, halo-(C₁-C₂)alkyl, (C₁-C₂)alkoxy, and (C₁-C₂)alkylthio;
one of R³ and R⁴ is hydrogen, and the other of R³ and R⁴ is selected from the group consisting of halogen, cyano, hydroxy, halo-(C₁-C₂)alkyl, hydroxy-(C₁-C₂)alkyl, and (C₁-C₂)alkoxycarbonyl; and
R⁵ is phenyl, wherein said phenyl is unsubstituted or is substituted with one or more groups selected from the group consisting of halogen, (C₁-C₂)alkyl, halo-(C₁-C₂)alkyl, (C₁-C₂)alkoxy, and (C₁-C₂)alkylthio;
or a pharmaceutically acceptable salt, tautomer or prodrug thereof.

33. The method of claim 28 wherein one or both of R¹ and R⁵ are phenyl substituents independently mono-substituted at the para position, and the R³ substituent is hydrogen.

34. The method of claim 28 wherein one or both of R¹ and R⁵ are phenyl substituents independently mono-substituted at the para position, and the R⁴ substituent is hydrogen.

35. The method of claim 28 wherein the compound is 1,5-bis(4-methoxyphenyl)-3-(trifluoromethyl)pyrazole, or the pharmaceutically-acceptable salts, tautomers and prodrugs thereof.

36. The method of claim 28 wherein the compound is a 3-(trifluoromethyl)-1,5-diarylpyrazole, or a pharmaceutically-acceptable salt, tautomer or prodrug thereof.

37. The method of claim 36 wherein the compound does not comprise a sulfonamide moiety.

38. The method of claim 28 wherein the compound is a 3-(difluoromethyl)-1,5-diarylpyrazole, or a pharmaceutically acceptable salt, tautomer, or prodrug thereof.

39. The method of claim 38 wherein the 3-(difluoromethyl)-1,5-diarylpyrazole does not comprise a sulfonamide moiety.

40. The method of claim 28 wherein the compound is selected from the group consisting of formulas: 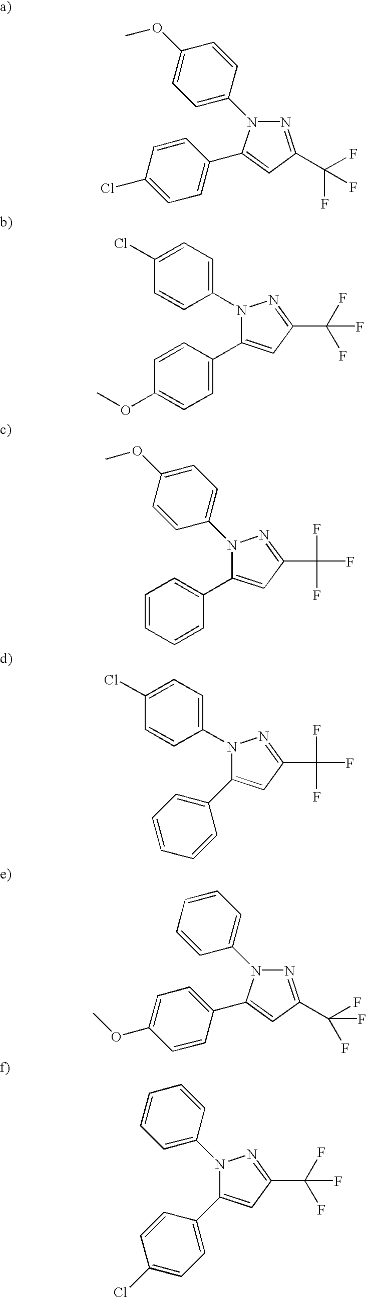 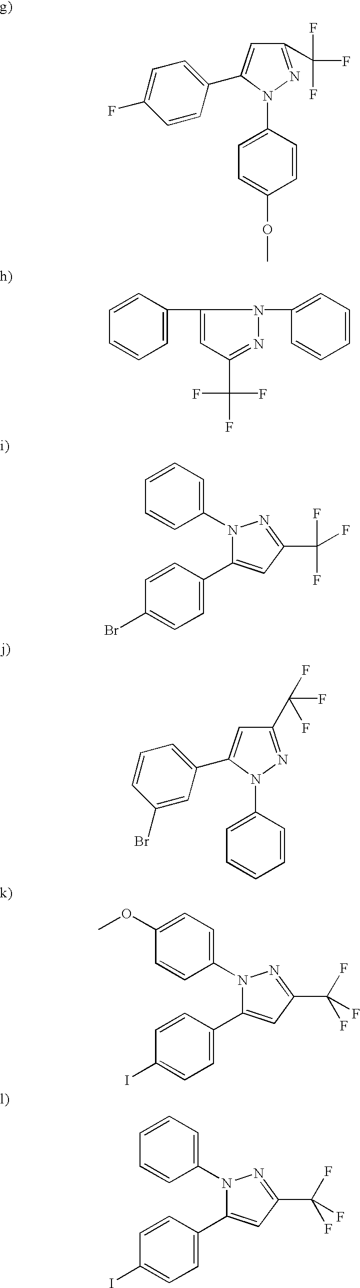 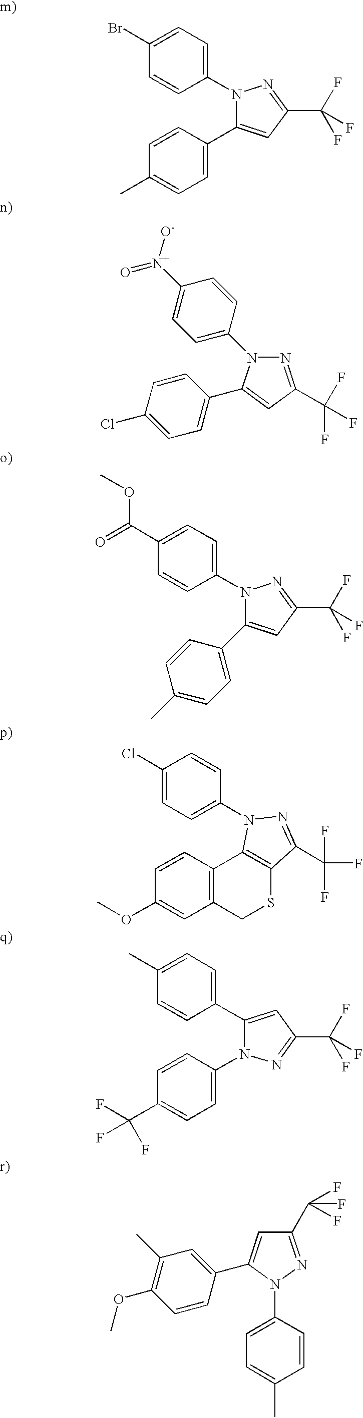 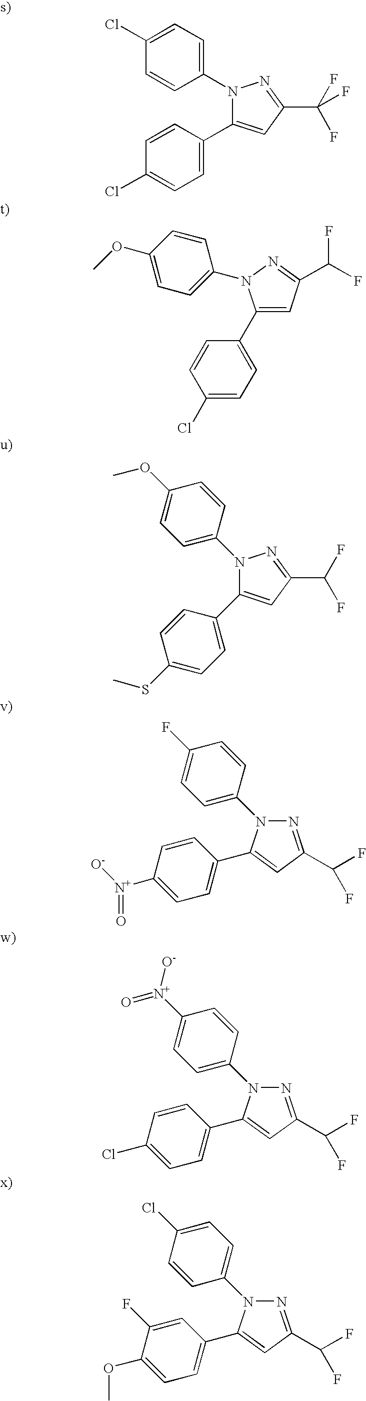 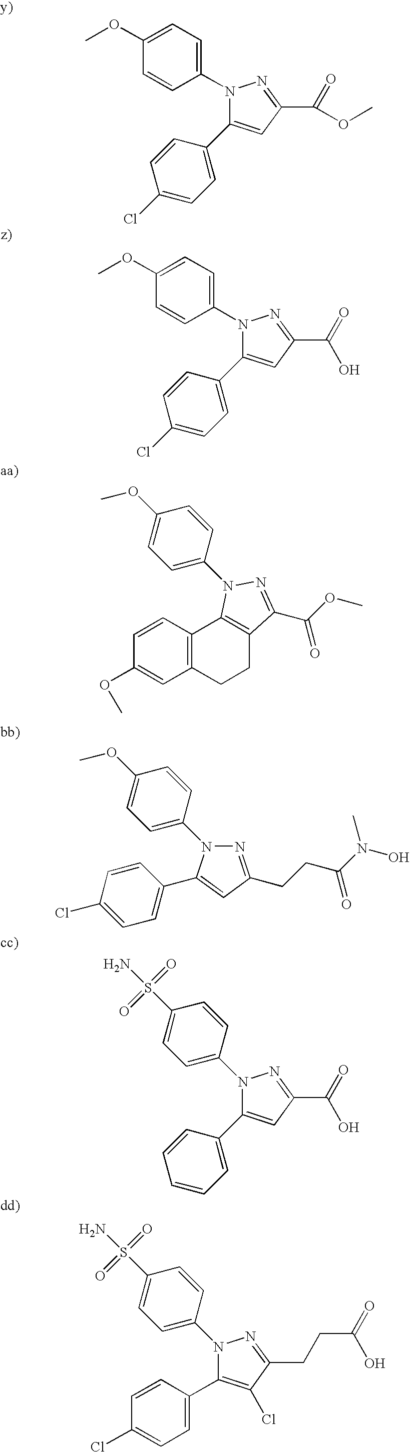 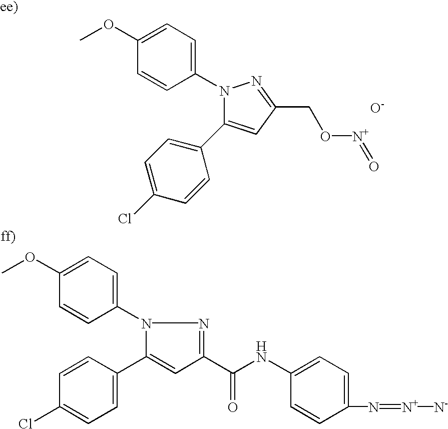
or pharmaceutically acceptable salts, tautomers, or prodrugs thereof.

41. The method of claim 27 wherein the cardiovascular condition is selected from the group consisting of coronary artery disease, aneurysm, arteriosclerosis, atherosclerosis including cardiac transplant atherosclerosis, myocardial infarction, aortic stenosis, renal dysfunction, renal failure, microalbuminuria, heart failure, embolism, stroke, thrombosis (including venous thrombosis), angina (including unstable angina), myocarditis, pericarditis, coronary plaque inflammation, bacterial-induced inflammation (including Chlamydia-induced inflammation), viral induced inflammation, auto-immune-induced inflammation, and inflammation associated with surgical procedures such as vascular grafting including coronary artery bypass surgery, revascularization procedures including angioplasty, stent placement, endarterectomy, and other invasive procedures involving arteries, veins and capillaries.

42. The method of claim 41 wherein the cardiovascular condition is atherosclerosis.

43. The method of claim 41 wherein the cardiovascular condition is thrombosis.

44. The method of claim 41 wherein the cardiovascular condition is restenosis.

45. The method of claim 41 wherein the cardiovascular condition is inflammation or hyperplasia resulting from vascular injury in a subject.

46. The method of any one of claims 27 wherein the cardiovascular condition is caused by or aggravated by cyclooxygenase-1 activity.

47. The method of claim 27 wherein the drug used in the treatment or prevention of a cardiovascular condition is selected from the group consisting of renin inhibitors, angiotension I receptor antagonists, angiotension II receptor antagonists, angiotension converting enzyme inhibitors, alpha-adrenergic receptor blockers, beta-adrenergic receptor blockers, calcium channel blockers, neutral endopeptidase inhibitors, aldosterone receptor antagonists, vasodilators, diuretics, lipid-lowering drugs, anti-oxidants, and IIbIIIa antagonists.

48. The method of claim 47 wherein the angiotension II receptor antagonist is selected from the group consisting of candesartan, eprosartan, irbesartan, losartan, and valsartan.

49. The method of claim 47 wherein the angiotension converting enzyme inhibitor is selected from the group consisting of alacepril, benazepril, captopril, ceronapril, delapril, enalapril, fosinopril, imadapril, lisinopril, moveltopril, perindopril, quinapril, ramipril, spirapril, temocapril, and trandolapril.

50. The method of claim 47 wherein the alpha-adrenergic receptor blocker is selected from the group consisting of amosulalol, arotinolol, dapiprazole, doxazosin, fenspiride, indoramin, labetolol, naftopidil, nicergoline, prazosin, tamsulosin, tolazoline, trimazosin, and yohimbine.

51. The method of claim 47 wherein the beta-adrenergic receptor blocker is selected from the group consistin of acebutolol, alprenolol, amosulalol, arotinolol, atenolol, befunolol, betaxolol, bevantolol, bisoprolol, bopindolol, bucumolol, bufetolol, bufuralol, bunitrolol, buprandolol, bubridine hydrochloride, butofilolol, carazolol, carteolol, carvedilol, celiprolol, cetamolol, cloranolol, dilevalol, epanolol, indenolol, labetalol, levobunolol, mepindolol, metipranolol, metoprolol, moprolol, nadolol, nadoxolol, nebivalol, nipradilol, oxprenolol, perbutolol, pindolol, practolol, pronethalol, propranolol, sotalol, sufinalol, talindol, tertatolol, tilisolol, timolol, toliprolol, and xibenolol.

52. The method of claim 47 wherein the calcium channel blocker is selected from the group consisting of bepridil, clentiazem, diltiazem, fendiline, gallopamil, mibefradil, prenylamine, semotiadil, terodiline, verapamil, amlodipine, aranipine, bamidipine, benidipine, cilnidipine, efonidipine, elgodipine, felodipine, isradipine, lacidipine, lercanidipine, madidipine, nicardipine, nifedipine, nilvadipine, nimodipine, nisoldipine, nitrendipine, cinnarizine, flunarizine, lidoflazine, lomerizine, bencyclane, etafenone, and perhexiline.

53. The method of claim 52 wherein the calcium channel blocker is selected from the group consisting of amlodipine, nifedipine, and verapamil.

54. The method of claim 47 wherein the neutral endopeptidase inhibitor is omapatrilat.

55. The method of claim 47 wherein the aldosterone receptor antagonist is eplerenone or spironolactone.

56. The method of claim 47 wherein the vasodilator is selected from the group consisting of cerebral vasodilators, coronary vasodilators, and peripheral vasodilators.

57. The method of claim 56 wherein the cerebral vasodilator is selected from the group consisting of bencyclane, cinnarizine, citicoline, cyclandelate, ciclonicate, diisopropylamine dichloracetate, ebumamonine, fasudil, fenoxedil, flunarizine, ibudilast, ifenprodil, lomerizine, nafronyl, nicametate, nicergoline, nimodipine, papaverine, pentifylline, tinofedrine, vincamine, vinpocetine, and viquidil.

58. The method of claim 56 wherein the coronary vasodilator is selected from the group consisting of amotriphene, bendazol, benfurodil hemisuccinate, benziodarone, chloracizine, chromonar, clobenfural, clonitrate, cloricromen, dilazep, dipyridamole, droprenilamine, efloxate, erythrityltetranitrate, etafenone, fendiline, floredil, ganglefene, hexestrol, hexobendine, itramin tosylate, khellin, lidoflaznve, mannitol hexanitrate, medibazine, nitroglycerin, pentaerythritol tetranitrate, pentrinitrol, perhexilline, pimefylline, prenylamine, propatyl nitrate, trapidil, tricromyl, trimetazidine, trolnitrate phosphate, and visnadine.

59. The method of claim 56 wherein the peripheral vasodilator is selected from the group consisting of aluminum nicotinate, bamethan, bencyclane, betahistine, bradykinin, brovicamine, bufeniode, buflomedil, butalamine, cetiedil, ciclonicate, cinepazide, cinnarizine, cyclandelate, diisopropylamine dichloroacetate, eledoisin, fenoxedil, flunarizine, hepronicate, ifenprodil, iloprost, inositol niacinate, isoxsuprine, kallidin, kallikrein, moxisylyte, nafronyl, nicametate, nicergoline, nicofuranose, nylidrin, pentifylline, pentoxifylline, piribedil, prostaglandin El, suloctidil, tolazoline, and xanthinolniacinate.

60. The method of claim 47 wherein the diuretic is selected from the group consisting of diuretic benzothiadiazine derivatives, diuretic organomercurials, diuretic purines, diuretic steroids, diuretic sulfonamide derivatives, diuretic uracils, amanozine, amiloride, arbutin, chlorazanil, ethacrynic acid, etozolin, hydracarbazine, isosorbide, mannitol, metochalcone, muzolimine, perhexiline, ticrynafen, triamterene, and urea.

61. The method of claim 60 wherein the diuretic benzothiadiazine derivative is selected from the group consisting of althiazide, bendroflumethiazide, benzthiazide, benzylhydrochlorothiazide, buthiazide, chlorothiazide, chlorthalidone, cyclopenthiazide, cyclothiaide, epithiazide, ethiazide, fenquizone, indapamide, hydrochlorothiazide, hydroflumethiazide, methylclothiazide, meticrane, metolazone, paraflutizide, polythiazide, quinethazone, teclothiazide, and trichlormethiazide.

62. The method of claim 60 werein the diuretic sulfonamide derivative is selected from the group consisting of acetazolamide, ambuside, azosernide, bumetamide, butazolamide, chloraminophenamide, clofenamide, clopamide, clorexolone, disulfamide, ethoxolamide, furosemide, mefruside, methazolamide, piretamide, torasemide, tripamide, and xipamide.

63. The method of claim 47 wherein the lipid-lowering drug is a statin.

64. The method of claim 63 wherein the statin is selected from the group consisting of mevastatin, lovastatin, simvastatin, pravastatin, fluvastatin, atorvastatin, rosuvastatin, and pitavastatin.

65. The method of claim 64 wherein the statin is atorvastatin.

66. The method of claim 47 wherein the anti-oxidant is vitamin E or probucol.

67. The method of claim 47 wherein the IIbIIIa antagonist is xemilofiban or orbofiban.

68. The method of claim 27 wherein the non-drug therapy used in the treatment of a cardiovascular condition involves exposure of an angioplastied artery at the site of injury to a source of radiation to inhibit restrictive neointima growth.

69. The method of claim 27 wherein the non-drug therapy used in the treatment of a cardiovascular condition involves the insertion of an endolumenal stent at the site of angioplasty.

70. The method of claim 69 wherein the stent is coated with any of the compounds of claims 1 to 14.

71. The method claim 47 wherein the selective cyclooxygenase-1 inhibiting compound and the drug used in the treatment or prevention of a cardiovascular condition are administered substantially simultaneously.

72. The method of claim 71 wherein the selective cyclooxygenase-1 inhibiting compound and the drug used in the treatment or prevention of a cardiovascular condition are combined and administered in the same dose.

73. The method of claim 71 wherein the selective cyclooxygenase-1 inhibiting compound and the drug used in the treatment or prevention of a cardiovascular condition are administered in separate doses.

74. The method of claim 47 wherein the selective cyclooxygenase-1 inhibiting compound and the drug used in the treatment or prevention of a cardiovascular condition are administered sequentially.

75. The method of claim 27 wherein the subject is a mammal.

76. The method of claim 75 wherein the mammal is a human.

77. The method of claim 76 wherein the human is at risk for developing an inflammation-related cardiovascular condition or has previously been diagnosed with an inflammation-related cardiovascular condition.

78. The method of claim 27 wherein the selective cyclooxygenase-1 inhibiting compound is administered to the subject in an amount of about 0.01 to about 100 mg/kg body weight per day.

79. The method of claim 78 wherein the selective cyclooxygenase-1 inhibiting compound is administered to the subject in an amount of about 0.5 to about 50 mg/kg body weight per day.

80. The method of claim 79 wherein the selective cyclooxygenase-1 inhibiting compound is administered to the subject in an amount of about 0.1 to about 20 mg/kg body weight per day.

81. A kit containing a first dosage form comprising a cyclooxygenase-1 selective inhibitor and a second dosage form comprising a drug used in the treatment or prevention of a cardiovascular condition.

82. The kit of claim 81 wherein the cyclooxygenase-1 selective inhibitor is a compound of Formula I: 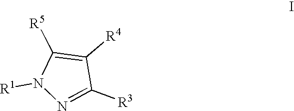
wherein:
R¹ is substituted or unsubstituted aryl;
one of R³ and R⁴ is hydrogen, and the other of R³ and R⁴ is an electron withdrawing group or a substituted or unsubstituted alkyl group; and
R⁵ is substituted or unsubstituted aryl;
or R³ is an electron withdrawing group or a substituted or unsubstituted alkyl group, and R⁴ and R⁵ combine to form a fused ring substituent;
or a pharmaceutically acceptable salt, tautomer or prodrug thereof.

83. The kit of claim 81 wherein drug used in the treatment or prevention of a cardiovascular condition is selected from the group consisting of renin inhibitors, angiotension I receptor antagonists, angiotension II receptor antagonists, angiotension converting enzyme inhibitors, alpha-adrenergic receptor blockers, beta-adrenergic receptor blockers, calcium channel blockers, neutral endopeptidase inhibitors, aldosterone receptor antagonists, vasodilators, diuretics, lipid-lowering drugs, anti-oxidants, and IIbIIIa antagonists.
